(19) United States
(12) Patent Application Publication
(10) Pub. No.: US 2005/0079498 A1
(43) Pub. Date: Apr. 14, 2005

(54) Novel Molecules

(76) Inventors: Cecilia Anders, Edmonton (CA); Barry Barton, Worthing (GB); Alison Michelle Griffin, Worthing (GB); Susan Jensen, Edmonton (CA)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/494,495

(22) PCT Filed: Nov. 6, 2002
(86) PCT No.: PCT/GB02/04989

(30) Foreign Application Priority Data
Nov. 7, 2001 (GB) 0126756.6
Nov. 30, 2001 (GB) 0128776.2

(51) Int. Cl. C12Q 1/68; C07H 21/04; C12N 9/10; C12P 37/00; C12N 1/21; C12N 15/74

(52) U.S. Cl. 435/6; 435/69.1; 435/193; 435/252.3; 435/320.1; 536/23.2; 435/43

(57) ABSTRACT

The invention relates to improvements in and relating to the biosynthesis of clavam compounds in particular clavulanic acid. Disclosed are new polynucleotides involved in the biosynthesis of clavulanic acid. Also disclosed are uses of said polynucleotides in improving clavulanic acid production.

The present invention relates to improvements in and relating to the biosynthesis of clavam compounds including clavulanic acid; to polynucleotides for use in improving or regulating the biosynthesis of clavam compounds and polypeptides encoded by such polynucleotides; and to the use of such polynucleotides in improving or regulating the biosynthesis of clavam compounds, in particular clavulanic acid, by a host cell.

Common to the structure of many important antibiotics, including all penicilins and cephalosporins, is a beta-lactam ring which is essential for their antibiotic activity. Degradation of the beta-lactam ring by beta-lactamase enzymes results in the loss of antibiotic activity. The ability of several micro-organisms to express beta-lactamases is therefore an important contributory factor in bringing about microbial antibiotic resistance.

Clavulanic acid is known to be a potent inhibitor of beta-lactamase enzymes (Reading, C and Cole, M (1977) Antimicrobial Agents and Chemotherapy 11 pp852-857), and has been successfully used in combination with beta-lactam antibiotics in drugs such as Augmentin (Registered Trade Mark), which includes clavulanic acid in the form of potassium clavulanate together with the beta-lactam amoxycillin, to combat infection by beta-lactamase-producing micro-organisms. Clavam compounds including clavulanic acid have thus become important pharmaceutical agents, and improvements in and relating to the production of such compounds are obviously desirable.

Clavulanic acid is produced by the gram-positive mycelial prokaryote Streptomyces clavuligerus, which also produces the beta-lactam compounds penicillin N, desacetoxy cephalosporin C and cephamycin C (Alexander et al, J. Bacteriol. (August 1998) Vol 180, No. 16:4068-4079). Research into the biosynthesis of clavulanic acid in Streptomyces clavuligerus has resulted in the identification and cloning of a 15 kb DNA fragment from S. clavuligerus which has been found to include nine complete open reading frames (ORFs) (designated orf2-orf10) which are involved in the biosynthesis of clavulanic acid (Canadian patent application CA 2108113). As is well known in the art an open reading frame defines a region of DNA that encodes a polypeptide. The open reading frame together with regulatory signals controlling expression of the polypeptide encoded thereby constitute a gene. Thus the 9 ORFs disclosed in CA 2108113 are believed to be the polypeptide coding regions of biosynthetic genes, each gene capable of expressing a polypeptide, for example an enzyme, involved in the biosynthesis of clavulanic acid in Streptomyces clavuligerus.

The functions of orf2-orf10, and/or the polypeptides encoded thereby (the orf2 to orf10 polypeptides are herein shown as the amino acid sequences of SEQ ID NO:14 to 22 respectively), were identified by biochemical analysis and/or sequence homology with known proteins. Orf5, for example, was found to encode the known enzyme clavaminate synthase II (Marsh et al, Biochem, 1992, 31:12648-12657), whilst orf2 was shown to possess a high level of homology with the enzyme acetohydroxyacid synthase (CA 2108113).

The enzymatic functionality conferred by orf2-orf10 has been compared against the metabolic pathway of clavulanic acid. It was found that each of the steps involved in the biosynthesis of clavulanic acid was capable of being catalysed by an enzyme encoded by the orf2-orf10 cluster; with the sole exception of the oxidative enantiomerisation of clavaminic acid to clavulanate-9-aldehyde, a step predicted to be mediated by a hydroxylase (Li et al, J. Bacteriol. (July 2000) Vol 182, No. 14, 4087-4095). This discovery prompted further investigation of the Streptomyces clavuligerus genome, which resulted in the identification of two further genes required for clavulanic acid biosynthesis: orf11 and orf12. Li et al (ibid) assigned putative functions to the products of the previously identified orf10 and the newly identified orf11, suggested that these genes were likely to be responsible for mediating the outstanding oxidative enantiomerisation step in the biosynthetic process (Li et al). Accordingly, it appeared that these genes completed the clavulanic-acid-producing cluster.

However the regulation and enhancement of clavulanic acid biosynthesis in clavulanic acid-producing hosts remains a desirable objective.

According to a first aspect of the present invention therefore, there is provided an isolated polynucleotide selected from the group consisting of:
a) a polynucleotide comprising a polynucleotide having at least 80%, preferably at least 90% homology, more preferably at least 95% homology, even still more preferably at least 97-99% homology, most preferably 100% 10 identity with the polynucleotide sequence of SEQ ID NO: 1 or with nucleotides 1 to 29744 of SEQ ID NO:1, over the entire length thereof;
b) a polynucleotide having at least 80%, preferably at least 90% homology, more preferably at least 95% homology, even still more preferably at least 97-99% homology, most preferably 100% identity with the polynucleotide sequence of SEQ ID NO: 1 or with nucleotides 1 to 29744 of SEQ ID NO: 1, over the entire length thereof,
c) a polynucleotide having the polynucleotide sequence of SEQ ID NO:1 or nucleotides 1 to 29744 of SEQ ID NO:1;
d) a polynucleotide which, by virtue of the degeneracy of the genetic code, encodes the polypeptides encoded by the open reading frames (ORFs) within SEQ ID NO:1, which polypeptides are herein identified as having the amino acid sequences of SEQ ID NO:8, SEQ ID NO:9, SEQ ID NO:10, SEQ ID NO: 1, SEQ ID NO:12, SEQ ID NO:13, SEQ ID NO:14, SEQ ID NO:15, SEQ ID NO: 16, SEQ ID NO: 17, SEQ ID NO: 18, SEQ ID NO:19, SEQ ID NO:20, SEQ ID NO:21 and SEQ ID NO:22.

Preferably the polynucleotide of the invention comprises a polynucleotide having the polynucleotide sequence of SEQ ID NO: 1 or having nucleotides 1 to 29744 of SEQ ID NO:1. Most preferably the polynucleotide has the polynucleotide sequence of SEQ ID NO:1 or nucleotides 1 to 29744 of SEQ ID NO: 1.

In view of the elucidation of what was believed to be the complete complement of genes required for clavulanic acid biosynthesis, as described hereinabove, it has therefore been surprising to find that the transformation of said polynucleotide into a clavulanic acid-producing host, such as wild-type S. clavuligerus, results in a significant increase in clavulanic acid yield.

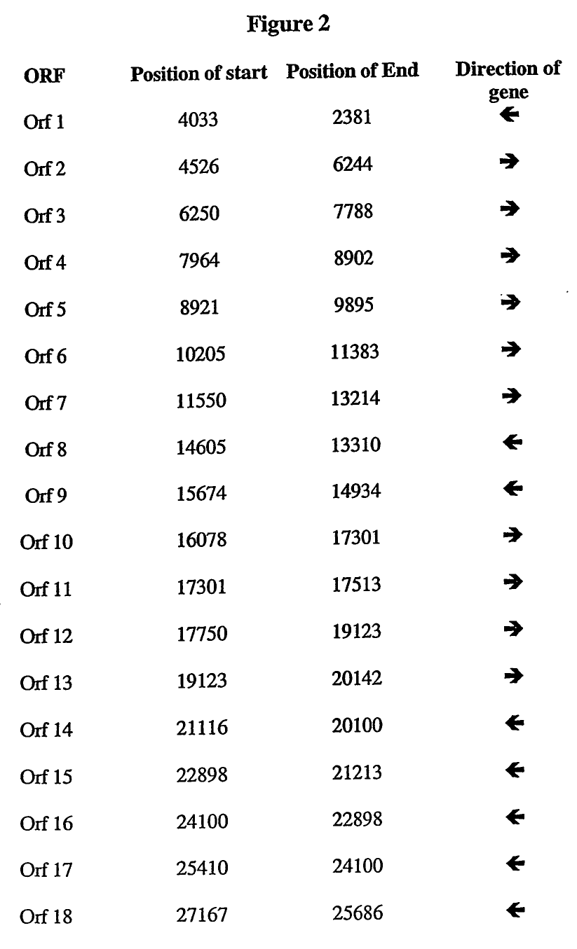

The polynucleotide of SEQ ID NO:1 was-first derived from a 36 kb fragment isolated from the genome of Streptomyces clavuligerus, a micro-organism which is conventionally used in the industrial biosynthesis of clavulanic acid. Sequence analysis and mapping reveals that the polynucleotide of SEQ ID NO: 1 includes the previously described orfs 2-10, 11 and 12, together with a sequence portion which extends downstream from orf 12. The sequence portion downstream from orf 12 has been found to include six further ORFs, here designated orfs 13-18 respectively. The nucleotide sequences for these six new ORFs are set out in SEQ ID NOs:2-7 respectively, whilst the polypeptide sequences encoded by each of these polynucleotides are set out in SEQ ID NOs:8-13 respectively. A table indicating the respective positions and orientations of each of orfs 2-18 in the polynucleotide of SEQ ID NO:1 is provided in FIG. 2 hereto.

According to a second aspect of the present invention therefore, there is provided an isolated orf 13, orf 14, orf 15, orf 16, orf 17, or orf 18 polynucleotide, which comprises or consists of an orf 13, orf 14, orf 15, orf 16, orf 17, or orf 18 nucleotide sequence that has:
(a) at least 80% homology, preferably at least 90% homology, more preferably at least 95% homology, even still more preferably at least 97-99% homology, most preferably 100% identity with the nucleotide sequence of a respective one of SEQ ID NO:2, SEQ ID NO:3, SEQ ID NO:4, SEQ ID NO:5, SEQ ID NO:6, or SEQ ID NO:7, over the entire length thereof, or
(b) at least 80% homology, preferably at least 90% homology, more preferably at least 95% homology, even still more preferably at least 97-99% homology, most preferably 100% identity with a nucleotide sequence which, by virtue of the degeneracy of the genetic code, encodes a polypeptide encoded by the open reading frame of SEQ ID NO:2, SEQ ID NO:3, SEQ ID NO:4, SEQ ID NO:5, SEQ ID NO:6, or SEQ ID NO:7, over the entire length thereof, or
(c) at least 80% homology, preferably at least 90% homology, more preferably at least 95% homology, even still more preferably at least 97-99% homology, most preferably 100% identity with a nucleotide sequence which encodes the amino acid sequence of a respective one of SEQ ID NO:8, SEQ ID NO:9, SEQ ID NO:10, SEQ ID NO:11 SEQ ID NO:12, or SEQ ID NO:13, over the entire length thereof.

According to a third aspect of the present invention, there is provided an isolated polynucleotide which comprises or consists of at least one of said orf 13, orf 14, orf 15, orf 16, orf 17 and orf 18 nucleotide sequences, and at least one of orf 2, orf 3, orf 4, orf 5, orf 6, orf 7, orf 8, orf 9, orf 10, orf 11 and orf 12 nucleotide sequences as disclosed in CA 2108113 and Li et al, ibid.

In one embodiment of this aspect of the invention, there is provided an isolated polynucleotide which comprises or consists of all of said orf 13, orf 14, orf 15, orf 16, orf 17 and orf 18 nucleotide sequences, and all of said orf 2, orf 3, orf 4, orf 5, orf 6, orf 7, orf 8, orf 9, orf 10, orf11 and orf12 nucleotide sequences.

Preferably, said polynucleotide comprises one or more promoter sequences for enabling the expression of at least one of said orf 13, orf 14, orf 15, orf 16, orf 17, orf 18 and, optionally, one or more of orf 2, orf 3, orf 4, orf 5, orf 6, orf 7, orf 8, orf 9, orf 10, orf 11 and orf12 in a suitable host.

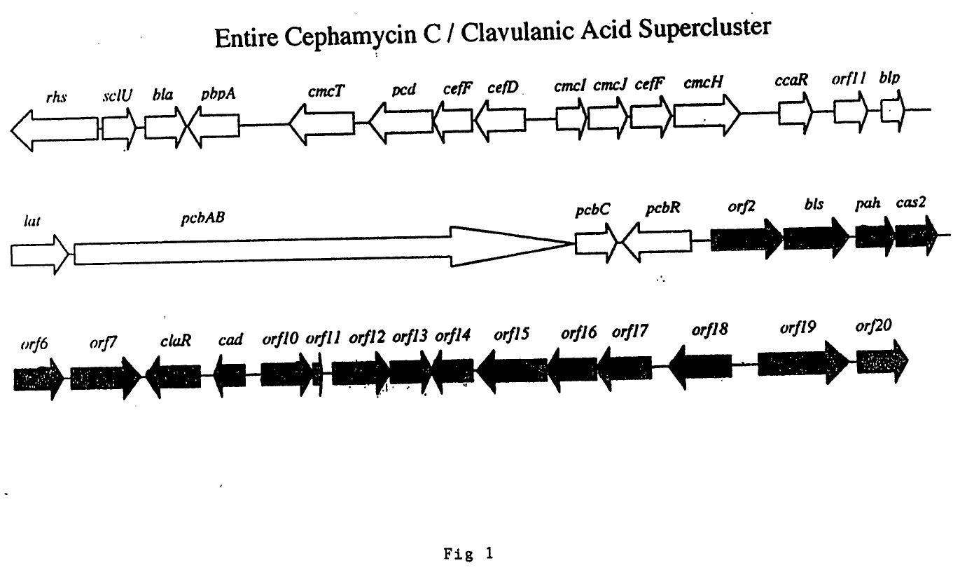

Advantageously, the orientation and relative arrangement of said orf 13, orf 14, orf 15, orf 16, orf 17, orf 18, orf 2, orf 3, orf 4, orf 5, orf 6, orf 7, orf 8, orf 9, orf 10, orf 11 and orf12 and/or said one or more promoter sequences may be identical or closely similar to the orientation and relative arrangement of said nucleotide sequences and promoter sequences in the genome of wild-type S. clavuligerus, for example as illustrated in FIG. 1 hereto, such that the transformation of said polynucleotide into a host will enable the expression of said polynucleotide sequences in said host

Analysis of the sequences of said ORF 13-18 polynucleotides has enabled the present inventors to ascribe the following putative functions to ORFs 13-18 respectively:

| SEQ ID NO | Gene name | Putative function |
|---|---|---|
| 2/8 | Orf13 | Transport protein |
| 3/9 | Orf14 | Acetyl transferase |
| 4/10 | Orf15 | Oligopeptide binding protein |
| 5/11 | Orf16 | oxidoreductase |
| 6/12 | Orf17 | Pyruvate enzyme/carbamoylphosphate |
| | | synthase |
| 7/13 | Orf18 | Resistance gene to intermediate/ |
| | | penicillin binding protein |

By “knock-out” analyses, described in more detail in Example 3 hereto, the present inventors have succeeded in showing that expression of each of said ORFs 13-18 is essential for, or required for, the efficient production of clavulanic acid in wild-type S. clavuligerus. Accordingly, a polynucleotide in accordance with any aspect of the present invention which consists of or comprises any one of said orf 13, orf 14, orf 15, orf 16, orf 17 and orf 18 nucleotide sequences, has potential utility in stimulating or enhancing the biosynthesis of clavulanic acid in a clavulanic acid-producing host, when expressed in said host.

According to another aspect of the present invention, there is provided an isolated orf 13, orf 14, orf 15, orf 16, orf 17, or orf 18 polypeptide which comprises or consists of an amino acid sequence having at least 80% homology, preferably at least 90% homology, more preferably at least 95% homology, still more preferably 97-99% homology, most preferably 100% identity with an amino acid sequence that is encoded by a polynucleotide of SEQ ID NO:2, SEQ ID NO:3, SEQ ID NO:4, SEQ ID NO:5, SEQ ID NO:6 or SEQ ID NO:7 respectively, over the entire length thereof. Advantageously, said isolated polypeptide may comprise or consist of the amino acid sequence of the respective one of SEQ ID NO:8, SEQ ID NO:9, SEQ ID NO:10, SEQ ID NO:11, SEQ ID NO:12 or SEQ ID NO:13.

A polynucleotide in accordance with any aspect of the present invention may comprise DNA or RNA. Suitably, said polynucleotide may comprise double-stranded DNA. Alternatively, said polynucleotide may comprise single-stranded DNA or single-stranded RNA.

Polynucleotides in accordance with the present invention may be prepared from a chromosomal DNA library prepared from S. clavuligerus or a related organism, utilising probe oligonucleotide sequences based on the sequences of said polynucleotides, in a manner well known in the art, for example as described in CA2108113. Alternatively said polynucleotides may be synthesised using well-established methods of polynucleotide synthesis, preferably using an automated DNA synthesiser.

According to a further aspect of the present invention, there is provided a vector which incorporates a polynucleotide in accordance with any aspect of the present invention. Said vector may advantageously be adapted to carry a large amount of exogenous DNA. Thus, said vector may for example be a cosmid vector, such as pWE15 or pLAFR3 (Staskawicz, B et al (1987) J. Bacteriol. 169 pp5789-5794). Alternatively, said vector may be a plasmid vector such as, for example, pTZ18R, pUC119, pBLUESCRIPTII SK+, pJOE829, pIJ702, pIJ922, pSL1180 or other plasmid or phagemid vectors known in the art Vectors suitable for this purpose are commercially available.

It will be appreciated that said polynucleotide may be inserted into said vector in either of two possible orientations, both of which are included within the scope of the invention.

According to yet a further aspect of the present invention, there is provided a recombinant cell comprising a vector according to the present invention. Such recombinant cells may be produced by the transformation of a host cell with a vector in accordance with the present invention, such that said polynucleotide incorporated in the vector can be expressed in said recombinant cell. Preferably the recombinant cell is a transformed Streptomyces spp host cell, for example Streptomyces clavuligerus or Streptomyces lividans. Most preferably the host cell is Streptomyces clavuligerus. Methods for the transformation of host cells are well known in the art and are described, for example, in Hopwood, D A et al (1985) Genetic Manipulation of Streptomyces. A Laboratory Manual (Ihe John lnnes Foundation); or Bailey, C R et al (1984) Biotechnology 2:801-811.

For effecting recombinant expression of said polynucleotide, a host cell can be genetically engineered to incorporate expression vectors or portions thereof for said polynucleotide. Introduction of polynucleotides into host cells can be effected by methods described in many standard laboratory manuals, such as Davis et al., BASIC METHODS IN MOLECULAR BIOLOGY (1986) and Sambrook et al., MOLECULAR CLONING: A LABORATORY MANUAL, 2nd Ed, Cold Spring Harbor Laboratory Press, Cold Spring Harbor, N.Y. (1989).

In some embodiments, said host cell may be adapted for the biosynthesis of clavulanic acid. Advantageously, said host cell may be a Streptomycete. Said host cell may, for example, be wild-type or recombinant S. clavuligerus, S. jumonjinensis, or S. katsurahamanus. A deposit of S. clavuligerus has been made at the American Type Culture Collection, Rockville, Md., USA under ATCC deposit number 27064 and the same strain has been deposited at the Agricultural Research Service Collection under deposit number NRRL3585 (Higgins C E and Kastner R E, (1971) Streptomyces clavuligerus sp. nov., a beta lactam antibiotic Producer Int. Journal of Systematic Bacteriology Vol. 21 No. 4:326-331). The NRRL3585 strain has now been redeposited on 5 Nov. 2001 by the present applicant at the National Collection of Industrial Food & Marine Bacteria (NCIMB), 23 St Macher Drive, Aberdeen, AB24 3RY, GB under accession number NCIMB 41121. A deposit of S. jumonjinensis has been made at the American Type Culture Collection, Rockville, Md., USA under ATCC deposit number 29864. A deposit of S. katsurahamanus has been made at the IFO collection under deposit number T-272 (Kitano K et al, (1979) Chem. Abstrac. 90:119758b).

Alternatively, said host cell may be recombinant strains of the genus streptomyces such as S. lividans, S. parvulus, S. griseofulvus, S. antibioticus, or S. lipmanii, which has been previously engineered to be capable of clavulanic acid biosynthesis.

According to yet another aspect of the present invention, there is provided a method for enhancing or stimulating the production of clavulanic acid by a host cell which is adapted to express clavulanic acid, comprising the steps of transforming said host cell with said vector, such that one or more polypeptides encoded by said polynucleotide can be expressed or over-expressed in said host cell, and culturing said host cell such as to allow production of clavulanic acid by the host cell.

Methods for culturing a clavulanic acid-producing organism so as to obtain clavulanic acid, and methods for purifying the clavulanic acid thus obtained, are set out in UK patent specification no. GB 1508977.

According to a further aspect of the present invention, there is provided a method for preventing clavulanic acid synthesis in a host cell which is adapted to express part or all of any one of the reverse complement sequences of said orf 13, orf 14, orf 15, orf 16, orf 17 or orf 18 polynucleotides, comprising the step of blocking the expression of said one of orf 13-18 polynucleotides in said host cell. Methods for gene-specific expression blocking are well known in the art and include for example the delivery of a single-stranded polynucleotide comprising part or all of the reverse complement of one of said orf 13, orf 14, orf 15, orf 16, orf 17 or orf 18 polynucleotides, such that said single-stranded reverse complement polynucleotide is enabled to bind to an mRNA transcript of said orf 13, orf 14, orf 15, orf 16, orf 17 or orf 18 polynucleotide and to block translation thereof. Alternative methods for gene-specific expression blocking include gene disruption or gene inactivation, as described in Aidoo, K et al (1994) Gene:147, 41-46 or Paradkar & Jensen (1995) J. Bacteriol (177) 5: 1307-1314; random mutagenesis and site-directed mutagenesis. Thus, said method may comprise the steps of preparing a vector incorporating an inactivated mutant orf 13, orf 14, orf 15, orf 16, orf 17, or orf 18 polynucleotide, introducing said vector to said host cell and culturing the host cell such as to permit inactivation, for example by a double cross-over recombination event with the corresponding wild-type orf 13, orf 14, orf 15, orf 16, orf 17 or orf 18 polynucleotide in the genome of said host cell. Said inactivated mutant polynucleotide may for example consist of an orf 13, orf 14, orf 15, orf 16, orf 17, or orf 18 polynucleotide having an oligonucleotide insertion or deletion such that said inactivated mutant polynucleotide does not encode an orf 13, orf 14, orf 15, orf 16, orf 17, or orf 18 polypeptide.

The following definitions are provided to facilitate understanding of certain terms used frequently herein.

“Isolated” means altered “by the hand of man” from the natural state. If an “isolated” composition or substance occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein, whether or not the polynucleotide or polypeptide is subsequently inserted into and/or expressed in a living organism. Thus, polynucleotides in accordance with the invention are not in their “natural” state, eg as found in the chromosomal DNA of S. clavuligerus, but are isolated from flanking chromosomal DNA.

“Polynucleotide” generally refers to any polyribonucleotide or polydeoxyribonucleotide, which may be unmodified RNA or DNA or modified RNA or DNA. “olynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising a plurality of amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Examples of such modifications may be found in, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttanslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Homology” is a measure of the degree of similarity of nucleotide sequences or amino acid sequences. In general, the sequences are aligned so that the highest order match is obtained. “Homology” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A M., and Griffin H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure homology between two polynucleotide or polypeptide sequences, the term “homology” is well known to skilled artisans (Carillo, H., and Lipton, D., SIAM J Applied Math (1988) 48:1073).

Methods commonly employed to determine homology or similarity between two sequences include, but are not limited to, those disclosed in Guide to Huge Computers, Martin J. Bishop, ed., Academic Press, San Diego, 1994, and Carillo, H., and Lipton, D., SIAM J Applied Math (1988) 48:1073. Methods to determine homology and similarity are codified in computer programs. Preferred computer program methods to determine homology and similarity between two sequences include, but are not limited to, GCS program package (Devereux, J., et al., Nucleic Acids Research (1984) 12(1):387), BLASTP, BLASTN, FASTA (Atschul, S. F. et al., J Molec Biol (1990) 215:403).

As an illustration, by a polynucleotide having a nucleotide sequence having at least, for example, 95% “homology” to a reference nucleotide sequence of SEQ ID NO: 1 is intended that the nucleotide sequence of the polynucleotide is identical to the reference sequence except that the polynucleotide sequence may include up to five base differences per each 100 nucleotides of the reference nucleotide sequence of SEQ ID NO: 1. In other words, to obtain a polynucleotide having a nucleotide sequence at least 95% homologous to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example, 95% “homology” to a reference amino acid sequence of SEQ ID NO:2 is intended that the amino acid sequence of the polypeptide is identical to the reference sequence except that the polypeptide sequence may include up to five amino acid alterations per each 100 amino acids of the reference amino acid of SEQ ID NO: 2. In other words, to obtain a polypeptide having an amino acid sequence at least 95% homologous to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

Variants of the defined sequences also form part of the present invention. Preferred variants are those that vary from the referents by conservative amino acid substitutions—i.e., those that substitute a residue with another of like characteristics. Typical such substitutions are among Ala, Val, Leu and Ile; among Ser and Thr, among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination.

In order to describe the invention more fully reference is made to Examples below and the accompanying drawings in which:

FIG. 1 shows the relative arrangement and orientation of ORFs 2-18 and surrounding genes in the genome of S. clavuligerus;

FIG. 2 shows the start and end points of ORFs 1-18 within SEQ ID NO:1;

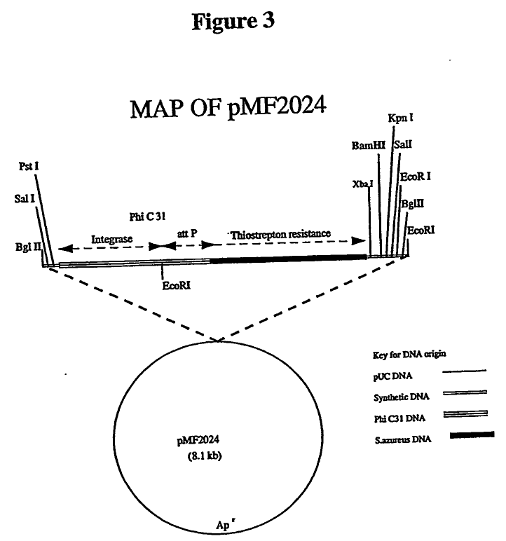

FIG. 3 shows a restriction map of vector pMF2024;

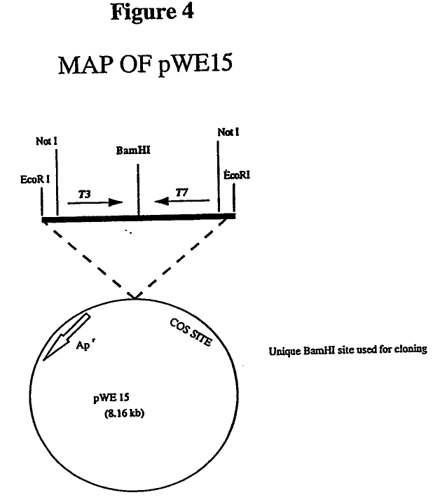

FIG. 4 shows a restriction map of vector pWE15;

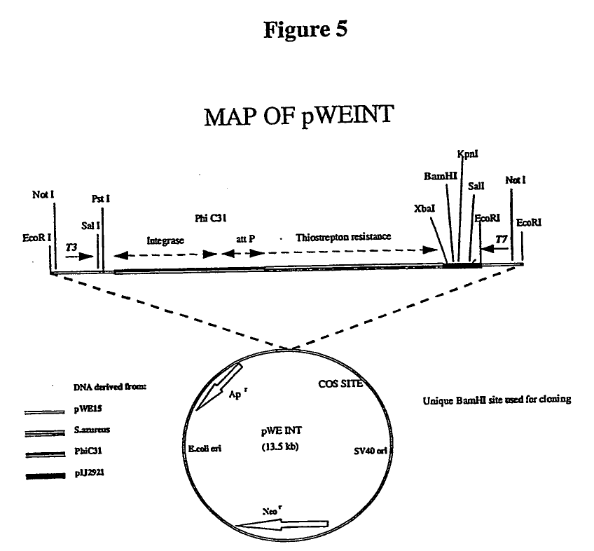

FIG. 5 shows a restriction map of vector pWEINT;

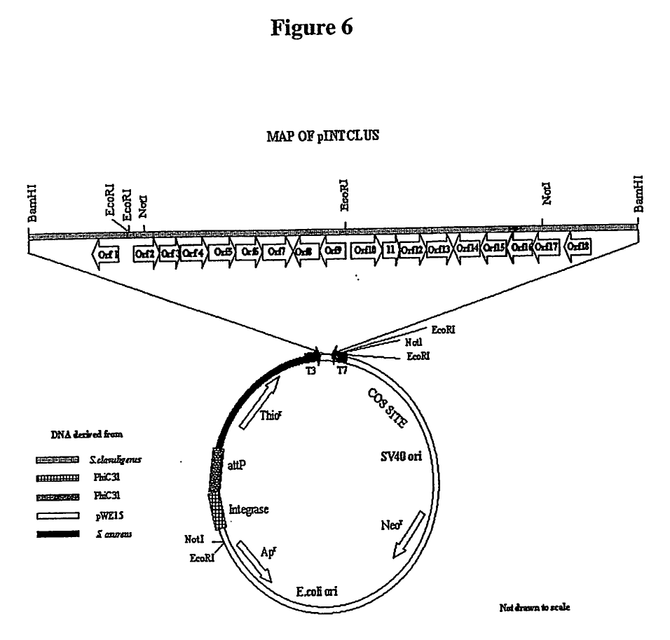

FIG. 6 shows a restriction map of gene cluster pINTCLUS.

### DESCRIPTIONS AND EXAMPLES

In the examples all methods are as in Sambrook, J., Fritsch, E. F. and Maniatis, T. (1989) Molecular Cloning A Laboratory Manual (2^{nd} Edition), or Hopwood, D. A. et al (1985) Genetic Manipulation of Streptomyces. A Cloning Manual, unless otherwise stated. In FIGS. 3-6 and the examples the abbreviations Bcl I, Bgl II etc are conventional abbreviations for restriction endonucleases. FIGS. 3-6 also give the approximate length in kilobases (kb) of the DNA. It should be understood that FIGS. 3-6 are not intended to show all the restriction sites present on the constructs illustrated.

### Example 1

1. Preparation of Chromosomal DNA from Streptonmyces clavuligerus ATCC 27064 Comprising orfs 2 ro 18

S. clavuligerus ATCC 27064 spores were used to inoculate a shakeflask of tryptone soya broth and maltose growth medium (25 ml/250 ml spring shakeflask—Tryptone soya broth 30g/l, maltose 10 g/l) and incubated for 48 hrs at 26° C. (with shaking at 240 rpm).

The mycelium was harvested by centrifugation at 3000 rpm for 10 minutes and washed in 10.3% sucrose. Chromosomal DNA was isolated using the “Total” DNA procedure 3. [Hopwood, D. A. et al (1995) Genetic Manipulation of Streptomyces, A Laboratory Manual, The John Innes Foundation]. 100 μg of the isolated chromosomal DNA was digested to completion with BamHI.

2. Size Fractionation of BAMHI Digested S. Clavuligerus ATCC27064 DNA by Sucrose Gradient Centrifugation

Sucrose gradients were generated as follows:— 3 mls of 40% sucrose in TEN (10 mM Tris HCL pH8, 1 mM Sodium EDTA, 1 mM NaCL) was placed into a 14 ml (14×95 mm), thin walled, polyallomer ultra tube. 3 mls of 30% sucrose in TEN was carefully layered on top of the 40% sucrose. 3 mls of 20% sucrose in TEN and then 3 mls of 10% sucrose in TEN were subsequently layered on top of the 30% sucrose. The 100 μg of BamHI digested chromosomal DNA was then loaded onto the gradient. The tubes were spun at 35,000 rpm for 16 hours at 17° C. using a swing out rotor (Sorvall TST 41.14).

500 μl fractions were taken from the tube starting at the top. To determine which fractions to use, 10 μl of each fraction was then electrophoresed on a 0.4% TBE agarose gel.

200 μl of TE (10 mM Tris pH8, 1 mM Na₂EDTA) was added to 200 μl of the appropriate fraction (fragments greater than 23 Kb). Carrier tRNA to a final concentration of 20 μg/ml was then added. The DNA was precipitated at room temperature for 10 minutes using an equal volume of isopropanol. The DNA pellet was rinsed with 100% ethanol, centrifuged and pellet resuspended in 20 μl TE.

3. Construction and Preparation of Cosmid Vector pWEINT

The integrative vector pWEINT (FIG. 5) was constructed using DNA from two sources, pMF2024 FIG. 3 and pWE15 FIG. 4. pWE15 is a commercially available vector from Stratagene (G Wahl (1989) Strategies 2 (17)) and pMF2024 was obtained from Paco Malpartida (University of Spain).

3 μg of pWE15 DNA was digested with BamHI under standard conditions until a sample electrophoresed on an agarose gel indicated that the digestion had gone to completion. The BamHI digested pWE15 DNA was cleaned up by phenol/chloroform extraction and precipitation with ethanol. The pellet was dissolved in 50 μl, of CIAP (Calf Intestinal Alkaline Phosphatase) buffer (Gibco BRL) and 1 unit of CIAP added. The DNA was then incubated at 37° C. for 30 minutes. A second unit of CIAP was added and the DNA incubated at 37° C. for a further 30 minutes. 45 μl of water and 5 μl of 10% sodium dodecyl sulphate (SDS) was added to the DNA and the DNA heat treated for 15 minutes at 68° C. The CIAP treated pWE15 DNA was then cleaned up by phenol/chloroform extraction and ethanol precipitation. The final pellet was dissolved in 10 μl of TE.

2 μg of pMF2024 was digested with BglII under standard conditions. The 5.4 Kb BglII fragment from pMF2024 containing the streptomyces bacteriophage φC31 integrase gene and attP site along with the thiostrepton resistance gene as a selective marker was isolated using the Pharmacia Sephaglas™ band prep kit (as per manufacturer's instructions).

10 μl of the isolated 5.4 Kb BglII fragment was ligated into 50 ng of Bam 1H digested, alkaline phosphatased treated pWE15 using standard protocols. The resultant ligation mix was used to transform Escherichia coli by standard methods. The transformed cells were plated onto L-agar plates supplemented with 50 μg/ml ampicillin. 80 transformants were obtained and patched onto L-agar containing 50 μg/ml ampicillin. Plasmid was isolated from 36 transformants using the boiling mini prep method (Holmes and Quigley 1981, Anal. Biochem. 144, 193). Restriction digest analysis with EcoRI confirmed that one isolate contained the 5.4 Kb BglII fragment from pMF2024 in pWE15: this construct is called pWEINT (FIG. 5).

4. Preparation of pWEINT Vector DNA

10 μg of pWEINT DNA was digested with BamHI until a sample electrophoresed on an agarose gel indicated that the digestion had gone to completion. The DNA was dephosphorylated using CIAP as described in Example 1.3. The CIAP treated DNA was resuspended in TE to give a final concentration of 1 μg/ml. Experiments were carried out to confirm the efficiency of the phosphatase reaction and the integrity of the BamHI cohesive termini (Statagene protocol for pWE15 and pWE16 cosmid vectors).

5. Ligation and Packaging of DNA

A final concentration of 225 μg/ml of DNA was used in the ligation reaction with the vector being present in a 10 fold molar excess. The size fractionated chromosomal DNA (>23 Kb from example 1) was ligated to BamHI digested pWEINT (Stratagene protocol for pWE15 and pWE16 cosmid vectors). For optimal packaging efficiency the Gigapack III Gold Packaging Extract was used Catalog # 200201/2 or 3. The packaging protocol outlined in the Gigapack III Gold Packaging Extract instruction manual was followed.

6. Titering the Cosmid Library

The Gigapack III Gold instruction manual was followed (Stratagene, Cambridge, UK). E. coli XL1-blue cells were transduced with the packaged cosmid library. 3×10⁶ E. coli XL1-Blue transformants (selected on L-agar plus 50 μg/ml ampicillin) were obtained per μg of DNA. Individual transformants (total of 960) were picked and grown in microtitre wells containing 125 μl of L-broth plus 50 μg/ml of ampicillin. The cultures were grown at 37° C. for 10 hours.

7. Preparation of Colony Blots

The Nunc TSP screening system (Life Technologies, Paisley, UK) was used to transfer cultures from the microtitre plate to Hybond N filters (Amersham International, Bucks, UK) which had been placed on L-agar containing 50 μg/ml ampicillin. The filters were placed in a 37° C. incubator overnight. The colony blots were then prepared as per the Amersham Membrane and Detection Methods (1985) p18. The filters were washed in 2×SSC, air dried and then wrapped in cling film. The wrapped filters were then placed colony side down on a U.V. transilluminator for 2-5 minutes.

8. Probing of Colony Blots

The filters were prehybridised in 200 mls of prehybridisation solution (6% PEG, 3×SSC, 1% SDS) and 20 mg salmon sperm DNA. Prehybridisation was carried out at 65° C. for 6 hours. The filters were then placed in Hybaid hybridisation bottles (300 mm×35 mm, Hybaid Ltd, Middlesex) as per manufacturers instructions. 30 mls of the prehybridisation solution was added to the bottle along with 5 mg salmon sperm DNA and α-³² PdCTP labelled probe. The probe used was a sub-fragment from pBROC44 (European Patent EP 0 349 121). 25 ng of this DNA was labelled using the Amersham Megaprime™ DNA labeling systems RPN 16041516/7. The hybridisation bottles were then placed at 65° C. overnight in a rotary Hybaid oven. The filters were washed at a high stringency (0.1×SSC at 65° C. for 1 hour). Autoradiographs were set up and exposed for 3 days at −70° C. The autoradiographs were developed using the X-ograph compact X2 automatic film processor. 16 positive signals were detected.

9. Plasmid Restriction Analysis and Sequencing

Plasmid DNA was prepared from the positive clones using boiling mini preps (Holmes and Quigley 1981 ibid). The plasmid DNA was digested with various restriction enzymes under standard conditions. Agarose gel electrophoresis confirmed that all the clones gave identical restriction patterns which were consistent with the cloned fragments being subfragments of the 60 Kb fragment shown in FIG. 1 of European Patent EP 0 349 121. One clone, carrying an approximately 36 Kb fragment (subsequently found to comprise the entire orf2 to orf 18 cluster) is called pINTCLUS (FIG. 6).

The sequence of 29744 bp of the 36 Kb fragment was determined using established techniques. This 29744 bp sequence contains the entire sequences of orf 2 to orf 18 and all the necessary natural expression control elements (eg promoters). An extended sequence of 29870 bp is given in SEQ ID NO:1. This extended sequence extends to and includes a natural BamHI site adjacent to orf 18 which is convenient for cloning.

10. Functional Analysis of the Open Reading Frames

Computer analysis of the DNA sequence shown in SEQ ID NO: 1 predicted the presence of a further 8 complete ORFS beyond those previously disclosed in Canadian patent application CA2108113. A description of each gene is shown in table 1.

| ORF DESIGNATION | Homology |
|---|---|
| ORF 11 | Ferridoxin |
| ORF 12 | β-Lactamase |
| ORF 13 | Transmembrane protein |
| ORF 14 | Transacetylase/tabtoxin |
| ORF 15 | oligopeptide binding protein |
| ORF 16 | RapaD oxidoreductase |
| ORF 17 | carbamoylphosphate synthase |
| ORF 18 | penicillin binding protein |

### Example 2

### Enhancement of Clavulanic Acid Titre in S. Clavuligerus by the Plasmid pINTCLUS

Plasmid pINTCLUS, containing the 36 Kb (orf 2 to orf 18) fragment, and prepared in accordance with Example 1 was transformed into S. clavuligerus ATCC27064 (Bailey, C. R et al 1986, J. Gen. Microbiol. 132, 2945-7). Thiostrepton resistant transformants were obtained and restreaked onto M5D (European Patent 0 349 121) medium plus thiostrepton (5 μg/ml). The transformants were then grown in shakeflasks for titre assessment. Spores from each isolate were inoculated into 20 ml of seed medium (European Patent 0 349 121) and grown for 3 days at 26° C. with shaking. 1 ml of the seed culture was then inoculated into a final stage medium (European Patent 0 349 121) and grown at 26° C. for up to four days with shaking. Samples of final stage broth were withdrawn after three or four days growth and assayed for clavulanic acid productivity as described in Bird, A. E. et al (1982) Analyst, 107:1241-1245 and Foulston, M. and Reading, C. (1982) Antimicrob. Agents Chemother., 22:753-762.

An increase in titre was observed in all the isolates tested with an average of a ten fold increase in titre over the parent clavulanic acid producer ATCC27064.

### Example 3

### Gene Disruption of orfs 11-18

To assess the possible roles of the open reading frames in the biosynthesis of clavulanic acid, insertional inactivation mutants were created by gene replacement. The basic method used for gene disruption and replacement was as described by Paradkar and Jensen (1995).

1. Cloning and DNA Sequencing of the Streptomyces Clavuigerus Chromosome Upstream of the orf 2 to 9 Clavulanic Acid Cluster

Cosmid clone K6L2 (isolated and characterised as described in CA 2108113) was digested with Pst I and EcoR1 restriction enzymes to generate a unique DNA fragment of 11.6 kb. This fragment was then ligated with plasmid pTZ18R (Pharmacia) also digested with Pst I and EcoRI restriction enzymes. The ligation mixture was used to transform E. coli XL1-Blue to ampicillin resistance. Two clones were isolated which possessed recombinant plasmids. These were confirmed by restriction analysis to carry the 11.6 kb fragment inserted within pTZ18R. This plasmid was named pCEC001.

Restriction analysis of K6L2 indicated that adjacent to the 11.6 kb DNA fragment cloned into pCEC001 there existed a 3.6 kb PstI-EcoRI fragment. Using the methods described above this 3.6 kb PstI-EcoRI fragment was subcloned from cosmid K6L2 into pUC119. The resultant recombinant plasmid generated was named p667-3.

2. Disruption of Orf11

To disrupt orf11 the plasmid pCEC002, which contains a 2.2-kb SphI-SphI fragment subcloned from pCEC001 into pBLUESCRIPTII SK+ carrying a small portion of orf10, all of orf11 and orf12, and some of orf13, was digested with BglII as pCEC002 possesses only one BglII site located almost exactly in the middle of orf11. The linearized pCEC002 was then treated with Klenow fragment and ligated to a Klenow-treated NcoI-NcoI fragment crying the apramycin resistance gene (apr). The ligation mixture was used to transform E. coli XL1-Blue to apramycin resistance. Two clones were isolated which possessed recombinant plasmids that were confirmed by restriction analysis to carry the apr^{r}-fragment inserted within orf11. One of the plasmids, pCEC041, carried the apr^{r}-fragment inserted into pCEC002 with the same orientation as orf11 This plasmid was then digested with BamHI and HindIII to release the insert and ligated with similarly digested pIJ486. The ligation was then used to transform Streptomyces lividans TK24 to apramycin and thiostrepton resistance. One clone was isolated and found to contain the recombinant plasmid pLOG221 (041 insert+pIJ486). Plasmid DNA pLOG221 was then used to transform wild-type S. clavuligerus. Apramycin and thiostrepton resistant pLOG221 transformants of wild-type S. clavuligerus were then subcultured to unsupplemented ISP medium #3 agar for two rounds of sporulation and were then replica-plated onto antibiotic-supplemented media. Putative double-crossover mutants, i.e. those that were apramycin resistant and thiostrepton sensitive, were obtained at a frequency of about 0.1% from pLOG221. Two putative mutants (221A, and 221B) were further characterised by Southern blot analysis. The results of the southern blot analysis confirmed that the chromosomal copy of the orf 11 gene had been disrupted as expected.

To test the effect of disrupting orf 11 on clavulanic acid biosynthesis seed cultures of 221A and 221B, prepared in trypticase soy broth supplemented with 1% maltose, were used to deliver a standardized inoculum to either starch-asparagine minimal medium (SA medium) or soya-flour (SF) medium. Supernatants, prepared from each culture at 24-h intervals, were analyzed for clavulanic acid production by HPLC and/or bioassay. All test cultures were compared to similarly grown wild-type S. clavuligerus reference cultures.

In SA medium, after 93 h growth, accumulations of clavulanic acid in mutants 221A and 221B were reduced by 23% and 66%, respectively. In SF medium, after 93 h growth, both 221A and 221B showed sharply decreased levels of clavulanic acid production (70-80%).

From these results it can be concluded that orf11 is required for efficient production of clavulanic acid biosynthesis and elimination of the gene by disruption causes a significant reduction in clavulanic acid production.

3. Disruption of Orf12

To disrupt orf12 pCEC002 was digested with EcoRI and NruI. The digest was Klenow-treated and then self-ligated and used to transform E. coli XL1-Blue to ampicillin resistance. The resulting transformants were screened for plasmid DNA and one clone was selected that contained pCEC036 in which a 400-bp EcoRI-NruI fragment, carrying one of the two BstEII sites found within pCEC002, had been deleted. The other BstEII site was located within orf12 at 659 bp from the start codon; pCEC036 was linearized with BstEII, blunted with Klenow fragment, and ligated to a Klenow-treated NcoI-NcoI apr^{r}-fragment. The ligation mixture was used to transform XL1-Blue competent cells to apramycin resistance. Restriction analysis confirmed that clones had been obtained with the apr^{r}-fragment inserted into orf12 in both orientations. In pCEC043 the apr -cassette is oriented in the same direction as orf12 but in pCEC044 it is oppositely oriented. The DNA fragments carrying the disrupted orf12 were freed from their respective plasmids by double digestion with BamHI and HindIII and ligated separately to similarly digested pIJ486. Both ligation reactions were the used to transform S. lividans TK24, however, only the pCEC044+pIJ486 ligation yielded apramycin and thiostrepton resistant transformants containing recombinant plasmids. One isolate from this transformation yielded the recombinant plasmid pLOG240. This plasmid was then used to transform wild-type S. clavuligerus. Apramycin and thiostrepton resistant pLOG 240 transformants of wild-type S. clavuligerus were then subcultured to unsupplemented ISP medium #3 agar for two rounds of sporulation and were then replica-plated onto antibiotic-supplemented media. Putative double-crossover mutants, i.e. those that were apramycin resistant and thiostrepton sensitive, were obtained at a frequency of about 2%. Two mutants (240-1C and -3A) were further characterised by southern blot analysis. The results of the southern blot analysis confirmed that in these mutants the chromosomal copy of the orf 12 gene had been disrupted as expected.

The mutants 240-1C, -2B, and -3A were then tested for their ability to produce clavulanic acid in SA and SF media as described in example 3.2. The results showed that none of the mutants were able to produce clavulanic acid when cultured in SA or SF medium for either 68 or 92 hrs.

The analysis of these gene disruption mutants indicates that orf12 is essential for clavulanic acid production.

4. Disruption of Orf13

To disrupt orf13 pCEC001 was digested with BstEII and a 2.9-kb fragment carrying both orf13 and orf14 and a small portion of orf15 was isolated, Klenow-treated and ligated to SmaI-isoested pBluescriptII SK+. The ligation mixture was used to transform XL1-Blue competent cells to ampicillin resistance; the transformants were screened for plasmid DNA and one clone possessing a recombinant plasmid, pCEC028, was isolated. The identity and orientation of the cloned DNA was confirmed by partially sequencing the insert using the T3 primer.

A Klenow-treated apr^{r}-fragment was ligated to NruI-digested pCEC028 which possessed a unique NruI site at 469 bp from the start codon of orf13 (approximately midway into the open reading frame). The ligation mixture was then used to transform XL1-Blue cells to apramycin resistance. Clones possessing the plasmid pCEC034 (containing the apr^{r}-fragment inversely oriented with respect to orf13) were isolated.

pCEC034 was then digested with Hind m and ligated with similarly digested pIJ486. The ligation reaction was then used to transform E. coli XL1 Blue. Those transformants which were able to grow on apramycin and thiostrepton were screened for the presence of recombinant plasmids. One isolate from this transformation yielded the recombinant plasmid pCEC047 (PCEC034+pIJ486). This plasmid was then used to transform wild-type S. clavuligerus.

Apramycin and thiostrepton resistant pCEC047 transformants of wild-type S. clavuligerus were then subcultured to unsupplemented ISP medium #3 agar for two rounds of sporulation and were then replica-plated onto antibiotic-supplemented media. Two putative double-crossover mutants, 47A3#3 and 47-1, i.e. those that were apramycin resistant and thiostrepton sensitive, were further characterised by southern blot analysis. The results of the southern blot analysis confirmed that in these mutants the chromosomal copy of the orf 13 gene had been disrupted as expected.

47A3#3 and 47-1 were analyzed for their ability to produce clavulanic acid in SA and SF media as described in example 3.2. The results showed that the ability to produce clavulanic acid in these mutants was reduced by up to 95% compared to the wild type control strain.

From these experiments, it can be concluded that orf13 is necessary for the efficient production of clavulanic acid.

5. Disruption of Orf14

To disrupt orf14, pCEC028 was digested with BalI; a BalI site is located within orf14 at approximately 160 bp from the translational stop codon. The BalI-digested pCEC028 was then Klenow-treated and ligated to a blunted NcoI-NcoI apr^{r}-fragment and used to transform XL1-Blue to apramycin resistance. Clones containing plasmid pCEC032 (with the apr^{r}-fragment inserted in the same orientation as orf14) were isolated.

A shuttle vector of pCEC032, was constructed by digesting the plasmid with HindIII and then ligating it with similarly digested pIJ486. The ligation was then used to transform protoplasts of S. lividans TK24 to apramycin resistance. The resulting apramycin-resistant transformants were subcultured to MYM agar supplemented with thiostrepton and apramycin and plasmid DNA isolated. The structure of the recombinant plasmids from these transformants were confirmed by restriction analysis of the plasmid DNA and confirmed that a pCEC032+pIJ486 hybrid plasmid had been isolated which was designated as pCEC046.

Plasmid pCEC046, was used to transform wild-type S. clavuligerus to thiostrepton and apramycin resistance. Three primary transformants were put through two rounds of sporulation under nonselective conditions as described for orf11 and putative disruptants were ultimately isolated from the progeny of each of the three primary transformants. One of these mutants 46-8a was flyer characterised by southern blot analysis. The results of the southern blot analysis confirmed that in this mutant the chromosomal copy of the orf 14 gene had been disrupted as expected.

The mutant 46-8a was grown in both SA and Soya-flour liquid and analyzed for its ability to produce clavulanic acid as described in example 3.2. The results from this experiment showed that this mutants was unable to produce clavulanic acid in either media at any of the time points tested.

Therefore from these experiments, it can be concluded that orf14 is essential for clavulanic acid biosynthesis.

6. Disruption of Orf15

The plasmid pCEC001 was digested with Nru I and a 4 kb DNA fragment was isolated containing part of orf13, all of orfs 14 and 15, and part of orf16. This fragment was then ligated with SmaI digested pBluescript II SK+ and the ligation mix transformed into E. coli XL1-Blue. On screening ampicillin resistant transformants the plasmid pCEC004 was isolated which contained the 4-kb NruI-NruI fragment from pCEC001 in pBluescript II SK+.

The BstXI site in the polylinker of pCEC004 was deleted in order to generate a clone with a unique BstXI site in orf15. In order to accomplish this pCEC004 was digested with SacI and XbaI, Klenow-treated, and self-ligated. The ligation mixture was used to transform XL1-Blue cells to ampicillin resistance. Transformants were screened for plasmids that were linearized by digestion with BstXI. A clone, containing plasmid pCEC037, was selected; restriction analysis confirmed that this plasmid consisted of a derivative of pCEC004 which had undergone an in vitro deletion in which the polylinker BstXI site had been removed.

The plasmid pCEC037 was then linearized by digesting with BstXI and blunted with T4 DNA polymerase. This DNA was then ligated to an apr^{r}-cassette that had been similarly blunted. The ligation reaction was used to transform E. coli to apramycin and ampicillin resistance. Transformants possessing recombinant plasmid pLOG101 was isolated; pLOG101 possessed the apr^{r}-cassette inserted in the opposite orientation as orf15.

To convert the plasmid pLOG101 into a shuttle vector the plasmid was digested with HindIII and ligated to HindIII digested pIJ486. The ligation reaction was then used to transform S. lividans TK24. Those transformants which were able to grow on apramycin and thiostrepton were screened for the presence of recombinant plasmids. Screening identified the shuttle plasmid pCEC063 which contained both pLOG101 and pIJ486. The plasmid pCEC063 was transformed into S. clavuligerus. Many thiostrepton-resistant apramycin-resistant transformants were obtained, out of which four were progressed further.

The four transformants were then put through two rounds of sporulation under nonselective conditions as described in example 3.2 and putative disruptants were identified by having a thiostrepton-sensitive apramycin-resistant phenotype. Three of these putative disruptants, 63-1A, 63-1B and 63-2A were further characterised by southern blot analysis. The results of the southern blot analysis confirmed that in these mutants the chromosomal copy of the orf15 gene had been disrupted as expected.

These disruptants were then analyzed for clavulanic acid production in both SA and Soya-flour liquid as described in example 3.2. The results from this experiment showed that all of mutants were unable to produce clavulanic acid in either media at any of the time points tested.

Therefore from these experiments, it can be concluded that orf15 is essential for clavulanic acid biosynthesis.

7. Disruption of Orf16

The plasmid pCEC001 was digested with Nco I and Sph I and a 5.4 kb DNA fragment was isolated containing part of orf13, all of orfs 14, 15, and 16, and part of orf17. This fragment was then ligated with Nco1 and Sph I digested pUC120 and the ligation mix transformed into E. coli XL1-Blue. On screening ampicillin resistant transformants the plasmid pCEC009 was isolated which contained the 5.4-kb Nco1-SphI fragment from pCEC001 in pUC120.

The plasmid pCEC009 was digested with Eco RI and ligated to plasmid pSL1180 (Escherichia coli phagemid vector, Pharmacia) that had been similarly digested with EcoRI. The ligation mix transformed into E. coli XL1-Blue cells and ampicillin resistant transformants selected. On screening these transformants the plasmid pCEC014 was isolated which contained the 5.4-kb Nco1-SphI fragment from pCEC009 in pSL1180.

The plasmid pCEC014 was digested with EcoICRI and BstX. The digest was fractionated by agarose gel electrophoresis and the resulting 5.9-kb fragment, carrying orf16, was eluted and purified. The fragment was treated with T4 DNA polymerase and self-ligated. Transformation of E. coli with the ligation mixture yielded a plasmid, pCEC065, which contained a unique NruI site within orf16.

To insert the apramycin resistance gene the apr^{r} gene was isolated from pUC120ApNco on an NcoI fragment, treated with Klenow to create blunt ends, and ligated to pCEC065 cut with NruI. Transformation of this ligation mix into E. coli yielded the plasmid, pCEC067, carrying the apr^{r} gene inserted into orf16 as expected. In the next step pCEC067 was digested with Hind III and ligated to pIJ486 digested with HindIII. The ligation reaction was then used to transform S. lividans TK24. Those transformants which were able to grow on apramycin and thiostrepton were screened for the presence of recombinant plasmids. Screening identified the shuttle plasmid pCEC068 which contained both pCEC067 and pIJ486. The shuttle plasmid pCEC068 was then transformed into wild type S. clavuligerus selecting for thiostrepton resistant, apramycin resistant transformants. Four primary transformants with this phenotype were then subcultured to unsupplemented ISP medium #3 agar for two rounds of sporulation and were then replica-plated onto antibiotic-supplemented media. Putative double-crossover mutants, i.e. those that were apramycin resistant and thiostrepton sensitive, were obtained at a frequency of about 2%-10%.

Four of these putative disruptants, 68-1A, 68-1B, 68-2A and 68-2D were further characterised by southern blot analysis. The results of the southern blot analysis confirmed that in these mutants the chromosomal copy of the orf 16 gene had been disrupted as expected.

These four disruptants were then analyzed for clavulanic acid production in both SA and Soya-flour liquid as described in example 3.2. The results from this experiment showed that all four mutants were unable to produce clavulanic acid in either media at any of the time points tested.

Therefore from these experiments, it can be concluded that orf16 is essential for clavulanic acid biosynthesis.

8. Disruption of Orf17

The plasmid pCEC001 was digested with Not I and HindIII and a DNA fragment of approx. 3 kb containing the orf17 was isolated. This fragment was then ligated with Not I and Hind III digested pBluescriptII KS+ and the ligation mix transformed into E. coli XL1-Blue. On screening ampicillin resistant transformants the plasmid pCEC062 was isolated which contained the 3 kb NotI-HindIII fragment from pCEC001 in pBluescriptII KS+.

The plasmid pCEC062 was partially digested with NcoI so as to obtain singly cut plasmid as the major digestion product The DNA fragments obtained upon partial NcoI digestion were then ligated with the NcoI-flanked apr^{r} gene, and introduced into E. coli, and apramycin-resistant ampicillin-resistant colonies were screened. Several plasmids were thus created in which apr^{r} had inserted into one of the three NcoI sites present on the plasmid. Screening of these plasmids identified plasmid pCEC072 that contained the apr^{r} inserted within the orf-17-NcoI site in an opposite orientation with respect to orf-17.

The plasmid pCEC072 was then digested with Hind III and ligated to pIJ486 digested with HindIII. The ligation reaction was then used to transform S. lividans TK24. Those transformants that were able to grow on apramycin and thiostrepton were screened for the presence of recombinant plasmids. Screening identified the shuttle plasmid pCEC076 that contained both pCEC072_and p11486.

The plasmid pCEC076 was then transformed into wild type S. clavuligerus selecting for thiostrepton resistant, apramycin resistant transformants. Transformants with this phenotype were then subcultured to unsupplemented ISP medium #3 agar for two rounds of sporulation and were then replica-plated onto antibiotic-supplemented media Putative double-crossover mutants, i.e. those that were apramycin resistant and thiostrepton sensitive, were obtained at a frequency of about 1%.

Several of these putative disruptants, 76-1A, 76-1B, 76-2A and 76-2B were further characterised by southern blot analysis. The results of the southern blot analysis confirmed that in these mutants the chromosomal copy of the orf 17 gene had been disrupted as expected.

These disruptants were then analyzed for clavulanic acid production in both SA and Soya-flour liquid as described in example 3.2. The results from this experiment showed that all mutants tested were unable to produce clavulanic acid in either media at any of the time points tested.

Therefore from these experiments, it can be concluded that orf17 is essential for clavulanic acid biosynthesis.

9. Disruption of Orf18

To assess the effects of inactivation of the orf18 gene upon clavulanic acid production an alternative strategy was undertaken to insert an additional copy of orf18 under the transcriptional control of the glycerol-inducible promoter into the φC31 attachment site of the S. clavuligerus chromosome prior to disrupting the wild type copy of the orf18 gene.

In order to construct a plasmid possessing the orf18 gene, which was under the transcriptional control of a glycerol-inducible promoter, a pair of synthetic DNA linkers were utilized in order to eliminate the need to perform a ligation involving two blunt ends. A XbaI-NaeI fragment containing orf18 originally from pCEC062 was ligated into compatible sites in the pSET based vector pMTX4 containing the gyl promoter (Smith, C. P. and Chater, K F. J. Mol. Biol. 204 (3), 589-580 (1988). The resulting construct, designated pMT8.34 was used to transform E. coli ER1447. Plasmid DNA that was purified from this strain was then used to transform wild type S. clavuligerus. Many apramycin-resistant transformants were obtained. The presence of p_{gyl}::orf18, which is presumed to have integrated at the ØC31 attachment site, was verified using PCR.

The orf18 disruption construct was engineered as follows. A unique EcoNI site, which is present midway between the two NcoI sites within orf18, was chosen as the target site for inserting the neomycin cassette. The plasmid pCEC062 was digested with EcoNI and treated with Kienow to create blunt ends and then ligated with the neomycin cassette isolated as an Acc651 fragment and also treated with Klenow, to create blunt ends. The ligation mixture was used to transform Ecoli and yielded pCEC084, which contains the neomycin cassette in opposite orientation with respect to orf18. pCEC084 was ligated to pIJ486 using SstI to yield the shuttle plasmid pCEC085.

Plasmid pCEC085, was transferred into E. coli ER1447, and from there into S. lividans. The digestion of plasmid pCEC085 with NcoI gave the same characteristic sizes of restriction fragments in all cases when it was purified from either S. lividans or E. coli ER1447. The transformation of the pgyl::orf18 strain of S. clavuligerus by pCEC085 yielded primary transformants that were neomycin, apramycin, and thiostrepton-resistant.

Transformants with this phenotype were then subcultured onto unsupplemented ISP medium #3 agar with 1% glycerol for two rounds of sporulation before replica-plating onto antibiotic-supplemented media Putative double-crossover mutants, i.e. those that were apramycin and neomycin resistant and thiostrepton sensitive, were obtained.

Southern blot analysis was carried out on these putative disruptants to determine whether the neomycin marker had inserted into the chromosomal orf18 or the pSET152 orf18. The results of the southern blot analysis demonstrated that in two of the mutants the chromosomal copy of the orf18 gene had been disrupted. One of these disruptants 1-5 was then tested for clavulanic acid productivity.

The disruptant strain 1-5 and the S. clavuligerus wild type strain NRRL3585 were fermented in SA as described in example 3.2 with the modification that additional fermentations were also set up with SA supplemented with 1% glycerol. In the SA fermentations the orf 18 gene is unable to be expressed from the gyl promoter as there is no glycerol present whereas in the SA fermentations where glycerol is added the orf18 gene is expressed from the glycerol promoter. Therefore by comparing the clavulanic acid productivity in these two different conditions it is possible to determine if the orf18 is involved in clavulanic acid production.

From these fermentations it was observed that clavulanic acid production by the disruptant strain 1-5 was reduced by 60% in SA fermentations compared to SA fermentations supplemented with 1% glycerol. For the wild type strain of S.clavuligerus no difference in clavulanic acid productivity was observed between the SA fermentations and the SA fermentations supplemented with 1% glycerol.

Therefore from these experiments, it can be concluded that although orf18 is not essential for clavulanic acid biosynthesis it is necessary for the efficient production of clavulanic acid.

1. An isolated polynucleotide selected from the group consisting of:
a) a polynucleotide comprising a polynucleotide having at least 95% homology with the polynucleotide sequence of SEQ ID NO: 1 or with nucleotides 1 to 29744 of SEQ ID NO: 1, over the entire length thereof;
b) a polynucleotide having at least 95% homology with the polynucleotide sequence of SEQ ID NO: 1 or with nucleotides 1 to 29744 of SEQ ID NO: 1, over the entire length thereof;
c) a polynucleotide having the polynucleotide sequence of SEQ ID NO: 1 or nucleotides 1 to 29744 of SEQ ID NO: 1;
d) a polynucleotide encoding the polypeptides encoded by the open reading frames (ORFs) within SEQ ID NO:1, wherein polypeptides have the amino acid sequences of SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21 and SEQ ID NO:22:

2. An isolated polynucleotide according to claim 1 which comprises a polynucleotide having the polynucleotide sequence of SEQ ID NO: 1 or having nucleotides 1 to 29744 of SEQ ID NO: 1.

3. An isolated orf 13, orf 14, orf 15, orf 16, orf 17, or orf 18 polynucleotide, comprising at least one open reading frame selected from the group consisting of orf 13, orf 14, orf 15, orf 16, orf 17, or orf 18 nucleotide sequence that has:
(a) at least 95% homology with the nucleotide sequence of a respective one of SEQ ID NO:2, SEQ ID NO:3, SEQ ID NO:4, SEQ ID NO:5, SEQ ID NO:6 or SEQ ID NO:7, over the entire length thereof; or
(b) at least 95% homology with a nucleotide sequence encoding a polypeptide encoded by the open reading frame of SEQ ID NO:2, SEQ ID NO:3, SEQ ID NO:4, SEQ ID NO:5, SEQ ID NO:6 or SEQ ID NO:7, over the entire length thereof; or
(c) at least 95% homology with a nucleotide sequence which encodes the amino acid sequence of a respective one of SEQ ID NO:8, SEQ ID NO:9, SEQ ID NO:10, SEQ ID NO: 1, SEQ ID NO:12 or SEQ ID NO:13, over the entire length thereof.

4. An isolated polynucleotide comprising at least one open reading frame selected from the group consisting of orf 13, orf 14, orf 15, orf 16, orf 17 and orf 18 nucleotide sequences, and at least one open reading frame selected from the group consisting of orf 2, orf 3, orf 4, orf 5, orf 6, orf 7, orf 8, orf 9 and orf 10 nucleotide sequences.

5. An isolated orf 13, orf 14, orf 15, orf 16, orf 17, or orf 18 polypeptide which comprises at least one amino acid sequence having at least 95% homology with an amino acid sequence that is encoded by a polynucleotide selected from the group consisting of SEQ ID NO:2, SEQ ID NO:3, SEQ ID NO:4, SEQ ID NO:5, SEQ ID NO:6 or SEQ ID NO:7, over the entire length thereof.

6. An isolated orf 13, orf 14, orf 15, orf 16, orf 17, or orf 18 polypeptide of claim 4 comprising at least one amino acid sequence selected from the group consisting of SEQ ID NO:8, SEQ ID NO:9, SEQ ID NO:10, SEQ ID NO:11, SEQ ID NO:12 or SEQ ID NO:13.

7. A vector which incorporates a polynucleotide of claim 1.

8. A recombinant cell comprising a vector of claim 7.

9. A recombinant cell according to claim 8 which is a transformed Streptomyces clavuligerus host cell.

10. A method for enhancing or stimulating the production of clavulanic acid by a host cell which is adapted to express clavulanic acid, comprising the steps of transforming a host cell with a vector according to claim 6, such that one or more polypeptides encoded by said polynucleotide can be expressed or over-expressed in said host cell, and culturing said host cell such as to allow production of clavulanic acid by the host cell.

11. A vector which incorporates a polynucleotide of claim 3.

12. A recombinant cell comprising a vector of claim 11.

13. A recombinant cell according to claim 12 which is a transformed Streptomyces clavuligerus host cell.

14. A vector which incorporates a polynucleotide of claim 4.

15. A recombinant cell comprising a vector of claim 14.

16. A recombinant cell according to claim 15 which is a transformed Streptomyces clavuligerus host cell.


SEQUENCE LISTING (22 sequences)

SEQ ID NO: 1 - DNA, 29870 bp, Streptomyces clavuligerus
gtggagggccacggccgggagcggttcgacggggcgcccgacgtccgggacacggtgggctggttcacgaccatgcacccgctggccgtggaggcgcacccgcaggacgtggggcgcggcatcaccgccacgaaggaggcttcccggcgggtgccgcaccacggcatcggatacggggccctcttcggccgctacggcggggagcgggcgccgctgccgccggtgagcttcaactacctcggccgggtctccgacgccggggagcagacggacgggccctccgcgggctggcggctcgactcggcgatgtcggggagcaacatctccggccgcaaccggggcgccgaccagttcagtgtggacgtgaccatgcgatgtaccggcggccggctcgtcaccgcggtggacagccgactggacgaggagaccacccggcgcttcacggacgagctgaggagctggctggagcggctcgtcgcccacacctcggcggtgtcgggcgagaacggcggccgggcgcgcgccgccgagcgggccggggccgggacggagttcgacccgtacatcctcgtcgacggggaccgcgccgagcgcaccctcttcgtcttcccgcccggtgagggcggcgccgagagctatctgagcaatctcgcgcagcggctgcccggccaccggctggtgctcttcaacaatgtgcatctgcaccggccgatggagtccttcgaggcgctggccgacttctatctccccctcctccgggagatccagccgtccggcccgtaccacctgctgggctggagcttcggcggggtgctctcggtggaggtctcgctccggctggcccgggcgggcgagcggatcgaggagctgttcctcatcgacccgtacttcgacgtccgggcctcgtcggcggcgatcgggctgccggagaccgaggacatcctcgatccgatcaactaccactgggcgcccgcccgggaggatctggagcggctgcgcgcgaacaccggcgatgtggtgctgttccgggcggacgagccgaacgagatcgtccgggacgaggagcagcgccggctgttcgacttctaccagcggtcctccttcaacgggctggacgcgctgctgcccgcggagtccatcgaggtccaccggctgcacggtgagacccaccattcgtgggtacgaaacgaccggctggtcgccgacatctgtgcgcgtgtctcggcgtcgtcgccggatgcccggtgaacggccgggcgctgagtccaaggagggtttcatgccagttctgatgccgtcggcccacgttccgaccatcgacatctcgccgctgttcggaaccgacgccgccgcgaagaagcgcgtcgccgaggagatacacggggcctgccgcggctcgggcttcttctacgccacgaaccacggcgtggacgtccagcagctccaggacgtggtgaacgagttccacggcgccatgaccgaccaggagaagcacgacctggcgatccacgcgtacaacccggacaacccgcatgtgcgcaacggctactacaaggcggtcccgggcaggaaggccgtcgagtccttctgttacctcaacccggacttcggcgaggaccacccgatgatcgccgcggggacgccgatgcacgaggtgaacctctggcccgacgaggagcggcacccgcgcttccggccgttctgcgagggctactaccggcagatgctgaagctctccaccgtgctcatgcgggggctggcgctggcgctcgggaggccggagcacttcttcgacgcggcgctcgccgagcaggactccctgtcgtccgtctcgctgatccgctacccgtatctggaggagtacccgccggtgaagacgggtcccgacggccagctcctgagcttcgaggaccatctggacgtctcgatgatcaccgtgctcttccagacccaggtgcagaacctccaggtggagacggtcgacggctggcgggacatcccgacgtcggagaacgacttcctggtcaactgcggtacctacatggcgcatgtcacgaacgactacttcccggcgcccaaccaccgggtgaagttcgtgaacgcggagcggctgtccctgccgttcttcctcaacggcgggcacgaggcggtcatcgagccgttcgtgccggagggcgcgagcgaggaggtgaggaacgaggccctgtcctacggggactacctccagcacgggctgcgggcgctgatcgtcaagaacggccagacctgagcggaaccggccgcccctgagcggggcggccgggaaggaaacgggccggtcgtcccctcgggagggggcggccggcccgtccggtgcgcgcggtgggtgcggcgcgggtcagccggccgcgaggttgctgaggaacttcgcggcgacggggcccgcgtcggcgccgcccgacccgccgtcctccagcaggaccgaccaggcgatgttccggtcgccctggtagccgatcatccaggcgtgcgtcttcggcggcttctcggtgccgaactcggcggtaccggtcttggcgtgcggctgtccgccgaggccccgcagggcgtcgccggcgccgtcggtgacggtcgaacgcatcatggaacgcagcgagtcgacgatgcccggggccatccggggggcctggtgcggcttcttgaccgcgtcgggcaccagcacgggctgcttgaactcgccctgcttgacggtggcggcgatggaggccatcaccaggggcgacgcctcgaccctggcctgtccgatggtggacgcggccttgtcgttctcgctgttggagacggggacgctgccgtcgaaggtggaggcgccgacgtcccaggtgccgccgatgccgaaggcttcggcggcctgcttcaggctggactcggagagcttgctgcgggagttgacgaagaacgtgttgcaggagtgggcgaagctgtcccggaaggtcgagcccgcgggcagcgtgaactggtcctggttctcgaagctctggccgttgacatgggcgaacttcgggcagtcggcccgctcctccgggttcatcccctgctggagcagggccgcggtggtgaccaccttgaaggtggagccgggcgggtagcggccctccagcgcgcggttcatgccggagggcacgttcgcggcggccaggatgttgccggtggcggggtcgacggcgacgatcgccgcgttcttcttcgagccctccagggccgccgcggcggcggactggacccgcgggtcgatggtggtcttcaccggcttgccctcggtgtccttgaggccggtgagcttcttgaccacctggccggactcacggtccaggatcacgaccgagcgcgccgcgccggagccgccggtgagctgcttgtcgtagcgggactggaggcccgccgagcccttgccggtcctggggtcgaccgtgccgatgatggaggcggcctggaggacattgccgttggcgtcgaggatgtccgcgcgctcccgcgacttgagggcgagggtctgccccggaaccatctgcggatggatcatctcggtgttgaacgcgaccttccactccttgccgccgccgacgaccttcgcggtggagtcccaggcgtactccccggccccggggagggtcattctgacggtgaacggtatctccacctcgccctcggggttcttctccccggtcttggcggtgatctccgtcttcgtcggcttgaggttggtcatgacggatttgatcagcgactcggcgttgtccggggtgtccgtcagcccggcggccgtcggggcgtcgcccttctcccaggcgccgaggaaggtgtcgaactgtccggccgccgcctccacctcggggtcgcccgaatccttctcgtcggcaaccaggctggtgtaaccccaatagccgagccccaccgtcacggccagcccggcgaccaccgcggtggccgcccggccacgggagcggcgcctgccctgcggcgagtcatcgccatagttgtcggaatgcgtcatggggccaggctatgcgggcgccctctttccctcctccccggataccgcgtttcaggacagtcaaggggccgaacggagggctggaccagccgctcagcggcccgttcccaccccttggggggaagcggcacccggaaggtgaccgaggcaacatccatggaaaggggagcgaatcggtcgccgagttcaccgcgattggagtagacctctgaaagcgtgacagcggggagtagcgacaaaacggtcagacccctgaagggaattgactgaattccatcgattgattgtgcgaatccagggaagccgagcgaattcgagtcatcgggttcggcgacggatgggcggttcggccacgcaccgtcactcttcgtcccctcttcacaagaactcccgatacgtggagaagagagcgtgaagagcgcgtccggtcagggttgccgagaaccgtccaccatgacggagcctggtactgacggagtctggagaccgctcatgtcccgtgtatcgaccgcccccagcggcaagcctaccgccgctcacgccctcctgtcacggttgcgtgatcacggtgtggggaaggtgtttggggttgtcggccgagaggccgcgtcgattctcttcgacgaggtcgaggggatcgacttcgttctgacccgccacgagttcaccgcgggtgtcgccgctgatgtcctcgcgcggatcaccggtcgcccccaggcgtgctgggccaccctgggccccggtatgaccaacctctccaccggtatcgccacgtccgtcctggaccgctcgccggtcatcgcgctcgccgcgcagtcggagtcgcacgacatcttcccgaacgacacccaccagtgcctggactcggtggcgatcgtcgccccgatgtccaagtacgccgtggagctccagcggccccacgagatcaccgacctcgtcgactccgccgtgaacgcggccatgaccgagccggtcgggccctccttcatctccctcccggtggacctgctcggctcctccgagggcatcgacaccaccgtccccaacccgccggcgaacaccccggcgaaaccggtcggcgtcgtcgccgacggctggcagaaggccgccgaccaggccgccgccctgctcgccgaggccaagcacccggtgctcgtcgtcggagcggccgcgatccgctcgggcgccgtcccggcgatccgcgccctggccgagcgcctgaacatcccggtcatcacgacctacatcgccaagggtgtcctgccggtcggccacgagctgaactacggcgccgtcaccggctacatggacggcatcctcaacttcccggcgctccagaccatgttcgccccggtggacctcgtcctcaccgtcggctacgactacgccgaggacctgcgcccgtccatgtggcagaagggcatcgagaagaagaccgtccgtatctccccgacggtcaacccgatcccccgggtctaccggcccgacgtcgacgtcgtcaccgacgtcctcgccttcgtggagcacttcgagaccgcgaccgcctccttcggggccaagcagcgccacgacatcgagccgctgcgcgcccggatcgcggagttcctggccgacccggagacctacgaggacggcatgcgcgtccaccaggtcatcgactccatgaacaccgtcatggaggaggccgccgagcccggcgagggcacgatcgtctccgacatcggcttcttccgtcactacggtgtgctcttcgcccgcgccgaccagcccttcggcttcctcacctcggcgggctgctccagcttcggctacggcatccccgccgccatcggcgcccagatggcccgcccggaccagccgaccttcctcatcgcgggtgacggcggcttccactccaacagctccgacctggagaccatcgcccggctcaacctgccgatcgtgaccgtcgtcgtcaacaacgacaccaacggcctgatcgagctgtaccagaacatcggtcaccaccgcagccacgacccggcggtcaagttcggcggcgtcgacttcgtcgcgctcgccgaggccaacggtgtcgacgccacccgcgccaccaaccgcgaggagctgctcgcggccctgcgcaagggtgccgagctgggtcgtccgttcctcatcgaggtcccggtcaactacgacttccagccgggcggcttcggcgccctgagcatctgatcatgggggcaccggttcttccggctgccttcgggttcctggcctccgcccgaacgggcgggggccgggcccccggcccggtcttcgcgacccggggcagccacaccgacatcgacacgccccagggggagcgctcgctcgcggcgaccctggtgcacgccccctcggtcgcgcccgaccgcgcggtggcgcgctccctcaccggcgcgcccaccaccgcggtgctcgccggtgagatctacaaccgggacgaactcctctccgtgctgcccgccggacccgcgccggagggggacgcggagctggtcctgcggctgctggaacgctatgacctgcatgccttccggctggtgaacgggcgcttcgcgaccgtggtgcggaccggggaccgggtcctgctcgccaccgaccacgccggttcggtgccgctgtacacctgtgtggcgccgggcgaggtccgggcgtccaccgaggccaaggcgctcgccgcgcaccgcgacccgaagggcttcccgctcgcggacgcccgccgggtcgccggtctgaccggtgtctaccaggtgcccgcgggcgccgtgatggacatcgacctcggctcgggcaccgccgtcacccaccgcacctggaccccgggcctctcccgccgcatcctgccggagggcgaggccgtcgcggccgtgcgggccgcgctggagaaggccgtcgcccagcgggtcacccccggcgacaccccgttggtggtgctctccggcggaatcgactcctccggggtcgcggcctgtgcgcaccgggcggccggggaactggacacggtgtccatgggcaccgacacgtccaacgagttccgcgaggcccgggcggtcgtcgaccatctgcgcacccggcaccgggagatcaccatcccgaccaccgagctgctggcgcagctcccgtacgcggtgtgggcctccgagtcggtggacccggacatcatcgagtacctgctccccctgacagcgctctaccgggcgctcgacgggccggagcgccgcatcctcaccgggtacggcgcggacatccccctcgggggcatgcaccgcgaggaccggctgcccgcgctggacaccgttctcgcgcacgacatggccaccttcgacgggctgaacgagatgtccccggtgctgtccacgctggcggggcactggaccacccacccgtactgggaccgggaggtcctcgatctgctggtctcgctggaggccgggctcaagcggcggcacggccgggacaagtgggtgctgcgcgccgcgatggccgacgccctcccggcggagaccgtcaaccggcccaagctgggcgtccacgagggctcgggcaccacgtcctcgttctcccggctgctgctggaccacggtgtcgccgaggaccgcgtccacgaggcgaagcggcaggtggtgcgcgagctgttcgatctcacggtcgggggcggacggcacccctccgaggtggacaccgacgatgtggtgcgctccgtggccgaccggaccgcgcggggggcggcctagtcccgccacggggagcccgccggacgccggacccgcgcgggacccgtacccggggccgcccgcggactccggcgcaccggcacccctgtcccccacccgttgacgaccgtcggccctcggccctcgcggcccctgacgaccgtcgcccgattcccaggagggagctgaaagcgtggagcgcatcgactcgcacgtttcaccccgctacgcacagatccccaccttcatgcgcctgccgcacgatccccagccccgcggctatgacgtggtggtcatcggagccccctacgacgggggcaccagctaccgtcccggcgcccggttcggcccccaggccatccgcagtgagtcgggcctcatccacggtgtcggcatcgaccggggccccggcacgttcgacctgatcaactgtgtcgacgccggggacatcaatctgacgccgttcgacatgaacatcgcgatcgacacggcgcagagccatctgtcgggcctgctgaaggccaacgccgcctttctgatgatcggcggcgaccactcgctgacggtggccgccctgcgcgcggtcgcggagcagcacggcccgctcgccgtggtgcacctggacgcgcactccgacaccaacccggccttctacgggggccggtaccaccacggcacccccttccggcacgggatcgacgagaagctgatcgacccggcggcgatggtccagatcggcatccggggccacaacccgaagccggactcgctcgactacgcccggggccacggcgtccgggtggtcacggcggacgagttcggcgagctgggggtgggcgggaccgccgacctcatccgcgagaaggtcggccagcggcccgtgtacgtctcggtcgacatcgacgtggtcgaccccgccttcgcccccggtacgggcacgcccgcgccgggcgggctcctctcgcgcgaggtgctggcgctgctgcgctgcgtgggtgacctgaagccggtcggcttcgacgtgatggaggtgtcacccctctacgaccacggcgggatcacttcgatcctggccacggagatcggtgcggaactgctctaccagtacgcccgagcccacagaacccagttgtgaaggagacatcgtgtcatggcctctccgatagttgactgcaccccgtaccgcgacgagctgctcgcgctcgcctccgagcttcccgaggtgccgcgcgcggacctccatggcttcctcgacgaggcgaagacgctggccgcccgtctcccggaggggctggccgccgctctcgacaccttcaacgccgtgggcagcgaggacggttatctgctgctgcgcgggctgcccgtcgacgacagcgagctgcccgagacgccgacctccaccccggccccgctggaccgcaagcggctggtgatggaggccatgctcgcgctggccggccgccggctcggtctgcacacggggtaccaggagctgcgctcgggcacggtctaccacgacgtgtacccgtcgcccggcgcgcactacctgtcctcggagacctccgagacgctgctggagttccacacggagatggcgtaccacatcctccagccgaactacgtcatgctggcctgctcccgcgcggaccacgagaaccgggcggagacgctggtcggctcggtccgcaaggcgctgcccctgctggacgagaagacccgggcccgtctcttcgaccgcaaggtgccctgctgcgtggacgtggccttccgcggcggggtcgacgacccgggcgcgatcgccaacgtcaagccgctctacggggacgcgaacgacccgttcctcgggtacgaccgcgagctgctggcgccggaggaccccgcggacaaggaggccgtcgcccatctgtcccaggcgctcgacgatgtgaccgtcggggtgaagctcgtccccggtgacgtcctcatcatcgacaacttccgcaccacgcacgcgcggacgccgttctcgccccgctgggacgggaaggaccgctggctgcaccgcgtctacatccgcaccgaccgcaatggacagctctccggcggcgagcgcgcgggcgacaccatctcgttctcgccgcgccgctgagcccggctccccgaggccctgggccccggcgccggaaccggctcccggtcctgccccctcacccgccgcgcgggtgagggggcaggcccctttgtgccgggtgccgtgcgtcctgcgagggtgccggggcgggggggacggcggaggtgcccggcggccgggtgccgtgcgccgcccgtgggtgctgtacagcactccgtgtgccgtgcgccaccccgtgcataaatttgccactctatgggaaataatgcagagtgcgacgggtgaggccgtcgccgtgccctttccgtgacaggagacgctgacatgtccgacagcacaccgaagacgccccggggattcgtggtgcacacggcgccggtgggcctggccgacgacggccgcgacgacttcaccgtcctcgcctccaccgccccggccaccgtgagcgccgtcttcacccgctcccgcttcgccgggccgagcgtcgtgctgtgccgggaggcggtggccgacgggcaggcgcgcggtgtggtggtgctggcccgcaacgcgaatgtcgcgaccggcctggagggcgaggagaacgcgcgcgaggtgcgcgaggccgtcgcccgggccctcgggctgccggagggcgagatgctgatcgcctccaccggggtgatcggccggcagtacccgatggagagcatccgggagcacctcaagacgctggagtggcccgccggggagggcggcttcgaccgcgcggcccgcgccatcatgacgaccgacacccggcccaaggaggtccgggtcagcgtcggcggggcgaccctcgtgggcatcgccaagggcgtcggcatgctggagcccgacatggcgacgctgctgaccttcttcgccacggacgcccggctggacccggccgagcaggaccgcctcttccgccgggtcatggaccgcaccttcaacgcggtcagcatcgacaccgacacctccaccagcgacacggcggtgctgttcgccaacggcctggcgggcgaggtcgacgccggggagttcgaggaggcgctgcacacggcggcgctggccctggtcaaggacatcgcgagcgacggcgagggcgcggccaagctgatcgaggtccaggtcaccggcgcccgcgacgacgcccaggccaagcgggtcggcaagaccgtcgtcaactccccgttggtgaagaccgccgtgcacggctgcgaccccaactggggccgggtcgccatggcgatcggcaagtgctcggacgacaccgacatcgaccaggagcgggtgacgatccgcttcggcgaggtcgaggtctatccgccgaaggcccggggcgaccaggccgacgacgcgctgcgggccgccgtcgcggagcatctgcggggcgacgaggtggtcatcgggatcgacctcgccatcgcggacggggccttcaccgtctacggctgcgacctcaccgagggctatgtccggctgaactcggagtacaccacctgatccccggacagggaacgggccgccgccccgttccctgtcgctcccgtcccgtgtggttataccgaccgttccccggctatgcgcacgggacggagcggcccccgccgggccccgcccggccgcacgatgaggggcgatgcaaggtgacgagggcaggagggacatggagaccactcggtcgacgaccgcggacgagggcttcgacgccggggtacggggagtggtcgcgccgaccgacgccccgggcgggacgctgcggctggtccgcacggacgacttcgactcgctcgaccccggcaacacgtactacgcctacacctggaacttcctccggctcatcggccggacgctggtcaccttcgacaccgcgccgggcaaggcgggccagcggctcgtgcccgacctcgccgagtcgctgggcgagtcctccgaggacggccgggtctggacctaccggctgcgcgagggcctgcgctacgaggacggcacgccggtcgtctcggccgacatcaagcacgccatcgcccgcagcaactacggcaccgatgtcctgggcgccggtccgacctacttccgccacctcctgggcaccgagtacggcggcccctggcgggagccggacgccgacggaccggtgacgctggagaccccggacgagcggacgctggtcttccggctgcgggagccgttcgcggggatggatctgctggcgaccatgccgtccaccacccccgtgccgcgcgaccgggacaccggcgccgagtaccggctgcggcccgtggcgaccggcccgtaccggatcgtctcgtacacccggggcgagctggccgtcctggagcccaatccgcactgggaccccgagaccgacccggtgcgcgtccagcgcgcctcccggatcgaggtgcacctcggcaaggacccgcacgaggtggaccgcatgctgctggcgggcgaggcccatgtggacctcgcgggcttcggtgtgcagcccgcggcccaggagcgcatcctcgccgagccggagctgcgcgcgcacgcggacaacccgctgaccggcttcacctggatctactgcctgtcgagccggatcgccccgttcgacaatgtgcactgccggcgggccgtgcagttcgccaccgacaaagcggccatgcaggaggcgtacggcggcgcggtgggcggcgacatcgcgaccaccctgctgcccccgaccctcgacggctacaagcacttcgaccgctacccggtcggccccgagggcaccggcgacctggaggccgcccgcgccgagctgaagctggccgggatgcccgacggcttccgcaccaggatcgccgcccgcaaggaccggctcaaggagtaccgggccgccgaggcgctggccgccgggctcgcccgggtcggcatcgaggcggaggtgctggacttcccgtcgggcgactacttcgaccgctacggcggctgcccggagtatctgcgcgagcacgggatcgggatcatcatgttcggctggggcgccgacttccccgacggatacggcttcctccagcagatcaccgacgggcgcgcgatcaaggagcgcggcaaccagaacatgggcgagctggacgacccggagatcaacgcgctgctggacgagggggcgcagtgcgccgacccggcgcggcgcgcggagatctggcaccgcatcgaccagctcacgatggaccacgcggtcatcgttccgtatctgtacccgcggtccctgctctaccggcacccggacacccgcaacgccttcgtcaccggctccttcgggatgtacgactacgtggcgctcggcgcgaagtgagcacggggtccggccccgggaccgtatgtcccggggccggaccccgcccgttccccgcccggtccggtccggacccggtcgcggcccgctcagccggacatccgggccccggccgcgaccccgcgccggatcggccagtggccctgcgccaggggccgttccacgctgcggcaggcgagagcggcctcgcggaactccgcctcgtacagcgcgagctggcgcaggaactgccgggtcgggccggtcaggctggtcccccgcgggctgcgcagcagcagccgggcgccgagggactgctccagccggtgaatccggcgggtgagcgccgactggctgatcgacagcaccgccgcggcccggttgatgctgccgtgccgggccacggcctggagcagatggagatcgtccacatccagtttgcggccctcggcctggccgggcacggagccctggtcgggtcccgccccgaagcggcgggcgtccgcgccggtgcgctccgcgtaccactgcgcccaccagggctcgtccagcaggtcgcggtggtgttcggcgaagcgccggagctggacctcggcgatcagcgcggccagccgtcccgccagcgcccggggcacgatggtggggtcgacgagcagactcgtggtgcggcgcgggcgctccgccagggagcggcgcaccagcgaggggtcctgcaccgccgggtgggtgggcgagccgagacctatcgcgtccccgcggcgcaggatgccccgggcaaccgatgcccccgtgatgtggagccgggtgggcgcggtgagcccggccagctggaagacacgtgtcaccaggatctccgagccgggtcccgtctcggacacccaggtctcgtcccgcagatcggcgagcgagacctcccgccgggcggccagcggatggtcccggggcaggatcacccacagcgggtcgtccagcacctcacaggtgcgcacggaccgctccaggctgtgccggggggactggaggctccaggtgtaggccgcgtccacctggtagcccgccagttgggcggcgacctggtgcggggcctcgtgccggaccgacagcagcaggtccagcgaggccgccgcgtcctccaccacctcgtcgagcaggggttccgtggagaccagcgacagcacctccggggcgtccacggcctcggagccatggccgaagatatgcgtccgcgcggccaggtcgacctggtggaagaaccgccgcccggcgacgaggatgcgggagcccgcggtggtcagccgggccgtgtggcggctgcgcagggtcagcgggaggccgacgatccggtccagccggtcgagtctgcgctccacggtgccgtgccggacacccgtccgccgggccacttccatgaggtctccgcagtgtcccaccgcgtccagtaaagacagatcgcatcggctgacaccagcagacgtcggttctgacccgagagacaatgtcggttcccttttccgtcaaggactgtaccgctgaattgtccgaagtggctcttgaattgcttcggaatcgatcctaggcagcgccgctcttcggattctcctcgccgggaagcggaacgcgcccggccggatggcgggcgcgctccgggcgccgtcccgggaacgggggacggggcacggcacggccggccacccggtccgggcgcgcggcgtggacctggtcggcggacgggtgtcagacctggtcggtggggcgtatgaagatctcgtggacggtcgcgtggtgcggcgcggtcacggcgtagcggaccgcctccgcgatgtcctgggcctggagcttgcggatctggctgatccgctgctcgtacatctccttggtggcggtgtgggtgatgtggccgcgcagctccgtgtcggtggtgcccggctcgatgacgacgacccgcaccccgcgctcggtgacctcctggcgcagcgtctcgctgaacgcgttcacaccgaacttcgtggcctggtagacggccgcgttgcggacgttcacccggcccgcgatcgaggacatctgcaccacggtgcccttgctgcgcagcagatggggaagggccgcccgggtcatgtacatcaggcccaggagattggtgtcgatcatccgggtccagtcggtggtgtcggcgtcctccaccgggccgagcagcatgatcccggcgttgttgacgaggatgtcgaggccgcccagcgcctcgacggtggaggcgacggcggcgtccaccccctgccggtcggcgacgtcgagttcgaggacatggaccttcgccccggcggcggtcagctcgtcacccagggcgcgcagcttctcgacccggcgcgcggcgatggccacggcggcgccctcggcggccagggcgcgggccgtggcctcgccgatgcccgagctcgcgcccgtgatgagcgcgactttcccctggagtgcggatggcatcatttcctccacatggtgctgcgatcgtggtgagcgtatgaagaaggggtgagacctgccgtgccggggcgggttccgtacgccggaccgttgcggtgggcacggccgaccgggtacggatggccgcagttccccggggagttcccggggaatggtgaataccgcggcgctctccgatggtcttcggaggacacccggggattcaccgggaatcagcggccggagttctccccgtccacggcagacgctatcagcgtcgcattccccggtgaattcccttcggtggaccgggttatgactgtttccgccgggttatgcgcgccgccccggcggaccggccacccgcccgggggctgcggcagattgggcgccacgacatggcgcgagcagcgatcggcggtggatgatgaacgaggcagcgcctcagtccgaccaggtggcaccggcgtatccgatgcaccgggtctgcccggtcgacccgccgccgcaactggccgggctgcggtcccagaaggccgcgagccgggtgacgctgtgggacggcagccaggtgtggctggtgacctcgcacgccggggcccgggccgtcctgggcgaccgccgcttcaccgcggtgacgagcgcgcccggcttcccgatgctgacccgcacctcccaactggtgcgcgccaacccggagtcggcgtcgttcatccgcatggacgacccgcagcactcccggctgcgctcgatgctcacccgggacttcctggcccgccgcgccgaggcgctgcgccccgcggtgcgggagctgctggacgagatcctgggcgggctggtgaagggggagcggccggtcgacctggtcgccggactgacgatcccggtgccctcgcgggtcatcaccctgctcttcggcgccggtgacgaccgccgggagttcatcgaggaccgcagcgcggtcctcatcgaccgcggctacaccccggagcaggtcgccaaggcccgggacgaactcgacggctatctgcgggagctggtcgaggagcggatcgagaacccgggcaccgacctgatcagccggctcgtcatcgaccaggtgcggccggggcatctgcgggtcgaggagatggtcccgatgtgccggctgctgctggtggccggtcacggcaccaccaccagccaggcgagcctgagcctgctcagcctgctcaccgacccggagctggccgggcgcctcaccgaggacccggccctgctgcccaaggcggtcgaggagctgctgcgcttccactccatcgtgcagaacgggctggcccgtgccgcggtggaggacgtccagctcgacgatgtgctcatccgggcgggcgagggcgtggtgctgtcgctgtcggcgggcaaccgggacgagacggtcttccccgacccggaccgggtggacgtggaccgcgacgcccgccgccatctcgccttcggccacggcatgcaccagtgcctgggccagtggctggcccgggtggagctggaggagatcctcgccgcggtgctgcgctggatgcccggtgcccggctcgcggtgcccttcgaggagctggacttccgtcatgaggtgtccagttacggcctcggcgccctcccggtgacctggtgagcggcgtggagcggctgaccgtcgtcctcgacgcgtcggcctgctgcgcgatggggcgctgcgcggccacggcccccgagatcttcgaccaggaccccgagacggggatcgccgtcctgctcgacgccactcccccgcccgagctgcacgagtcggcccggctctgtgccgagctgtgcccgtgcgaggccatcacggtcaccgagggctaggtcgtgtcgtcaaaatcccgtctgcgtcgcgacgccttgcacgcacatctgccgcgttgtcgtcagtcgccgatgctccgcatggactccctcctccgccttgcatctgcacgcaccatgcgccgcaccgcccgtcctccggacggacgacgctattttgacgacactcccttgacagccttggcgccgttccgcgcgcctcccgcaccgtggccgagggccgacaaggagcgatgatgaagaaagctgattccgtcccgaccccggctgaggcggccctcgccgcccagaccgcgctcgcggccgacgactcccccatgggcgacgcggcccgctgggcgatggggctgctgacctcgtccggactgccccggccggaggatgtggccgcgcgcttcatccccaccttcgcggcggcggggaacttcgcggagaccgtgcgggagtggcggtcgaagggccccttcacggtccgggcctaccaccccgtcgcgcacaagggctgggtcgtgctgtcggccccggccggggtgcggtacatcctgtcgctgacgctggactccagcgggctgatccgcatcctcaccctgaagcccgagaccgtcatacccgacatggtcacgtggaacgatgtcgaggagaccctccacaccccgggtgtgcagcactccgtgtacgcggtgcggctgacgcccgacgggcacgaggtgctgcacgcgtcggcgccggagcggccgatgccgaccgggtcggcgtacaagctctatctgatgcgcgcgcwcgtcgccgagatcgagaaggggacggtgggctgggacgagatcctcacgctcacgcccgagctgcgcagcctgcccacgggcgatatgcaggacctccccgacggcacccgggtgaccgtgcgggagaccgcgcacaagatgatcgcgctgagcgacaacaccggcgccgatctggtcgcggaccggctgggccgggaggtcgtggagcgctccctggcagccgcgggccaccacgacccgtccctgatgcgcccgttcctcaccagccacgaggtctttgagctgggctggggcgacccggagcggcgcgcggagtgggtccggcaggacgaggcgggccgccgggagctgctggagaagatggccggggtgatgaccgtccgcggctccgacctgggcgcgacggtccatcagctcggcatcgactggcacatggacgccttcgacgtggtccgggtgctggaggggctgctccaggacagcggacgggacaccagcggcacggtcgaggagatcctcaccgcctaccccggtctgctgatcgacgaggagcgctggcgccgggtctacttcaaggccggttccagccccggtgtgatgatgttctgctggctgctccaggaccacgcgggcatctcgtacgtcctcgtcctgcggcagtcggccgacgagcagcggctcatcggcgacggtctgttcctgcgcgggatcggcgccaagatcatcgaggccgaggcgaagctgctcagctccggtgagcgccgcggtgccgggaccgcggcggccggggacgaccgcgcgagcgccggagaagccgcccggcgatgagcgtcagagaccggctcctcgcctgcttcgtggcggtgctgtggggcctgaacttcctcgccgtgcgcatcggcctcgactactaccctccggtgttcctctccgcgatgcggttcgtggtcgtggccgtgccggtgatcctcttcgtgccccggcccaaggtgcccctgcgctggctgctggtgtacggactgggcttcggtgtgatccagttcgggctgctcttcctcgccatcgacatcggcatgccctcggggcaggcgtccgtggtggtgcaggcggcggccccgttcaccatgctgctcgggctgctgctgggggagcgcatctcccggcggcaggcgggcggcatcgtgctggccgtggccgggatgagcgccatcgcggtggaacgggcgcaggacgcggccctgctgccgctggtgctgaccctggtggcggcgttcggctgggcgatgggcaatatcgcgagccggcaggcccgcccggaccatccgctgcgcttcgcgctctggatgtgtgtgctgccgccgatcccgctgctgggcctgtcggccgccctggagggcccgacggcgggctggcgggccctcggcgactcgctcacctccggcgacctcaccggtccggtggcgctgctgtacacggccctcgcgggctccgtggtcggctccgggatctggaacaccctgatgaaacggtacgaggcgggcaccgtggcgccgttctcgatgctggttccggtggtcgccgtcgcggtcgccacgatctggctggacgagcggctgaccctctggtcggcggtgtccggcgcggccgtggtgctgggcgtgctggtggggacgacccggtcgcgcgcggcccgggaggcgggcccgcccccggccgccggaaggccgccgaaggccccggcggaacgcggccggatgctgcggcggcgcccaccggggcccggggacacgggtcccgatcagtcggacggaccggcctcggcggtccagaacacccagcggtcgtagggctcgaagcccagcgcggcgttgacggcgatcatcgggtggttgtcctcggcgttcgccgtctccacgagccgcacctccggctcgtgccgcagcacgtactccagattggccagcttcagcagggttcccagggcgtggccgcggtgctcacggtgcaccacggtcatgccctggagcgcgtacgcggggttcccggtggtcttggagacgctggtgtacccggcgagagcgccggtggcgtcgtggacggcgccggtgtggtacgcccgccggccgcggccgacccgcatggtctcgaactgccgggcgtagctcgtcctgacctcctgcgcggccctgtcgacgggcccggcgccgagcgagagttccagctcggagaccggcaccgcgtactcgtcgggggtgatggtgccccaggtgacgagggagtacccggcggggaccgcggggacgccgtccgccagcgggtcgtgccggtcgaggtcgagccactggtgggtgcccgccgggatgtccgagcggtgcgcgcccatcgcggcggcgaaggcggccgggcccgggtcctgggccgggccggagggcagggactcgacgacggtcgcggtgagggtggtccggtcgtgcttgcgggccagttcccgggcgtgggcccagagcgcgcggccgatgccgcggcggcgccgcccggggtggaccaggagctggtcgacgcgggcggtcggggcgccgtcgggcagggccagccgcagcgcgcccaccacccggcccccgctgcggacgacccagtcgtcgagggcggtcgcgggcggggcgaagcgcagcgagccgaccatgtcgacattgcaggggggcgcggcccgggggccggtctcggcggcgagggccatcaggtcgagccactggccgaccgtttcgtcgtcgttcgccgcgagggggacgaactccagttcgccggcggtgtcgttcatgcgggtcccttgtgagtcggtcgggttcggtgccgcgggccgggcccggcgggggacgggtgcgccgtcccccgccgggcccgggtgatggacctagtcttcttcggcgaggcccatcgcctggatgtcgtagaggccgaaggccgggtggacatagacgttggtgacccacgggttgcggaagtgcagcgtcttgtcgtggacgaggggcagcagcacggcgtgctccatcacccggcgctccacggcccgccacagctccgcgcgcgcggcggggtcggtggtgtgcagggtctcgtcgatcagcgcgttgacctcggggtcgtccagctcgggcaggttccagttgccgccgttgcgcttgatctggcggccgtccacgagcggggcgaggaagccgtactcggtggggaagtcggcgccccagtcggtgacgaggagtccgagcccgtgctcccggacggtctcggggtgcccggcgccgaggctgaagtaggtggcgacgtcgagttccttgacggtcagctcgatgccgacccgggcgagcgactccacgaccgcgtcggcgaccagccggaacttgccccgctgggtgccgatcaccgcccggaagccgtccgggaggcccgccgccgccagctcggcccgcgccgcgtccaggtcgccgcggaggtccggccccgtggggtacagatccaggtcctggtgggcggggagggtgggcgggaagagcgccgtggtcaggtcgccgccgttgaccgggccgccgcgggcgtcctggagcaggatcttgtccgccgcgtactgcaccgcgcggcgtacgtgcacattgtcgaacggggggatgtgcggctgcatcgcaacgaagtggaggaagctcgtccgggggttgtccgtgtgcgagcgcagcacctcgtccgcggtggcccggcgctgggccgcgtgctggagcccccggccctccaggttgatgtcgaactccccggcgatcagccgggcgtccaggacatcgacgtccagaccgatggtcagctccacccggtccggcagcgcgggcctgatcgggtccgtcgcccggtcccagtgcggattgcgctccaggtggagcagggtgtccggctcatggcgggcgacccggtaggggcccgacgagcgcgggtcgcggccgtagtcggcgccggtgtccgagcgccggggcacgggcgccgcgcagggctgggccatcagatggtcgaagtcggagaacggccgggtcagccggaagacgatcgtgtgctcgtcgggggtgaggaccgaccgcagcggctcgtccgtccggtacggaccggggtagggccgctcggggtcgtccagcagcgggatgagataggtggggccgccgggcagcacgtcctgcgcgaacacccgctgcacggcgtggcgcacatcgtccgacgtgatcggggtgccgtcgtcgtagcgcagcccccggcgcagccggtaggtccaggtccggccgccgtcgctgacctcgccggggccctcggccaggtccggggccgggaccagtccggcggggccggggtcggtcggataggccatgagcgtacggttcagcagccgctggagcagccacacccagacgtagtaggtgcgggccgggtcgagggagtcgacgtcggccgaggagacgagccgcagggtcccgccgcgccgccgcgaggggttcaccagcgcccccaccccggcgtcccagccggctccggcggtcgtcggggccggtcgtcgcgcggcggtggtcatggtgcggtcctcttctgatcggtgtcggtggtgccggtctccccggtgcgcggcaggcgcagcaggaacgagtgctcctggctggccagacagccgcgggccagcttcgcctcgacgctgccgatcccggcgtgcagcacggtcacgccctcgcgggcggcggcctccagcacccggtagtacaggacgtcgtagtagagcggcagcctgccctgggcctcgtagtcgaatccggcgcggtggccgaaccactcctcgcggccccgggcccgggagcgcagcaccatgccgaagccgatgatcaccccgtccttgcgggccacggagaccatcacgtcgtcgcccatgacctgggcgatccagctcagcagccccgcggagtgctcggggctcgccgggttgccgtacttgcgcagcagattgcagtcgagttcgccgagccggggcacgatctcctgggtcagcggctcgatccgcacctcgatgcccgccgcgtccagcgcccggcgctccagccggacccggcggcggcggtgcttggtcatgtcggccaggaactcgtcccagccgccgggcgggatgtcgagccaggcgtagtgcgccgaggggtgcgagacatagccgcgcgagcgcagcagcccgatcagggcgccgtcgcggtcgtcgacgtgcgggaagcagacggtggccgcgccccgctccttcgcgagccgctcggcctcggcgaccagcgcctccacgtcggacggcagcgcgtcgggcgcggcgagcgggcgggtgcggcccagatgccggccgccgcacaccagcgagggcagcagcgaggcggggtctcccccggccgtgtcggcgagccgggcctcggcctcggggtgctccccctcgccctccagcgcccgggtgagcatcgcgtccgggcgggccagcagccagggcacggagtcgtcgccccacgcggtcaccagggccgcgacgggacggcccgcgcggcggcggaccaggaagtccatcgtggtgccggagttgcgctcctgcaccgccagccagcggggcgtctggaagaagtcctccgggccgacgagatccggccagccgctggtggggagggcggcggcgtcgggcaccgccaccacgtccgtcaccgcgtccgccgccgtgtgcggcagcgcgtccgtcaccggcagggtggcgtctccgctcacgactccccgccccgctcgccccgctcgtcctcctcgacgacgaagtccaggtatccggccgcgcggtccgcgagggcctgggcctcctcccggtccgccccggcgcagtaggtgaagccgacgcacgaggagttgtccatgcgctggagcagcttgtcgcccttctccacgtgcagcagcacctggaccccgggcaccgcggccgcggcctcgatgccgtcgacccgggtgagggtgcccttccgggtcgggtagatcatgcgcacggcggcgccgccgttgtggacgacgggctcgatcggcggggcctcgccgatcgtcagcagccaggcgtcgaggatcgggctgcgcccggtgacatcggtgaccagcatcgtcatacagctcgacggggtgcgcgggttggcctcgatgatccgccagccggtctcggtgcgccgcacctcggtgtggcaggggccgtgccggtagccgatcgcctccagggtgccccggacggcggcgtacagcgcctcgcgctcctcgtcggagaggggcaccgggaaggtgtgcccgcactcgatgtacgcggggtcgccgaacaggtccttggaggtgaccccgaagacatgggtgtacccgtccacggtcatcgtctcgacgctgacctcgacgccctcgacataggtctccaggaggacatcgccggagcgcggctggccgcgcaggctgtggcggacgccggacagctcctcgtacgcctcgaccagctccgccatggtgtcgacgcggcgcaccccgtagctgatggcctcggcgggcggcttggcgatcatggggaagccgagcttccgtacggcctcgtcgagctgctcgcggctgctgacccgggcgtgcgggatgttgtaggggacgtcgccgagggcgaggcgcagccggtccttctcgttcgccgtctccacggcctcggggtcggggccgggcagcgacagccgctgggccgcctgcgcggcgatgaccgtgtggtagtcggagaacgaggtgagcccgtcgaccaggcactgcccgctctcgtcggtgacggccgcgagcacggccgccagatcggtggtgtccacgtcgaccacccggtcgacggccgccgcgagggggtggctgtcccagtcgtgcccgtgggtgtaccaggaccggtccccggtgagcagccagacctcaaggccgtgctcacgtgcctggtggagccccaggacgaaggggtggttctgcatgtgctcgacgatcgcgatgcgcttgcccgcgaacttggcatagtcggccaggggtgtggtcatcgaaccgcctccgtcatgacgctgttcagtcgactcgactcggatacacaggctccagttgaccgcgccgatcacgttcgggacacggttacggacgcggtgtgcataggctgtgagcgaggcgcataggaaggcggccggcggggcccgcgcgggccgcgcggcatgccggaaaagcccgatggctcccgccctcggggggtgggagccatcgtcgtcggaaagccgggccgcggggcccgggaacgggccgggggccggggccccgggggtcacttctccaggacggccttcatcacactgcgggcgatgggcgcgcccagaccgccgccggagatgtcctcacgggagatgtccatcgcggtcgggtcgatgaagacggccaccgccacggactggccgtccttctccccgtaggagacgaaccagccgtacgggacctgctcgcgcacatcgacaccgcgctgcgcggtaccggtcttgccgcccaccgtgacgccgtcgatcagcgccttgcgggcgctgccctccctggcggtgaactccatcatctcccggaccttgcccgcggtctcctcggagacggcctggctcatctcccggggctcgttcttctccagggtgctcagatcgggggactgcacctcgtcgacgatgtacggctgcatcagcttgccgttgttggcgatggcggcggtgaccatggccatctgcaagggggtgctggtgaggctgccctgtcccataccggtcagcgcggtgcccggcttgtccagttccttggggtacaggctcttgacggcccgcatgtcgccgaaggcgtccgagtagacgtcctcgttgaacccgaacttctcggcggtgtcgcgcatcttgtccacgcccagcttgaccgcggcgtcgaggaagacattgttgcaggaatgctgcatggcggtcttcagcgagaccttgtcgcaggccgcgttgcccgcctcgctgccgaccgtgttggtcgactgcggcagcggatacggggacaccgcgtcggtcttcgcgtcgatgtcgtcgatcaccccgtgctccagggccgccgccgccgtgaggatcttgaacgtggagccgggcgggaaggtctcccgcagcgcgcggttggccagcggcttgcccttgtccttgtccaggtcccggaaccgcttgctctccttgaggctgttgcccgcgaagacaccggggtcgtaggacggggtcgagaccagggcgagcaccttgcccgtcttcgggtcgagcgccacggccgagccccgggcgtccagggcggtgagccccttgtacgcggcctgctgcgccttggggtcgatcgtggtgatcacattgccgccgcgccggtccttgccggtgacgacgtcggtgaagcgctggatcgcgagccggtcgtcctgcccgctcaatacgctgtcgaacgtgttctccagcagcgacatgcccatggactgcgaggcgtacccggtgacgggcgcgtacatggggccgttcttgtaggtgcgctggtacttgaagtcggtgccgtcgacggccttcgacccggtgatggaggtaccgccacccacgatgatgtcgccgcgcggggtcgcgaactggttgatctggacccggcggttgcgctcgtgcgtggccagttcctcggcgtccacgtactgcagccagttggctctcagcagcagcgcgagtgtcagcagcccgcagaagatggctatgtgccgcagcggcctgttcatggagcacctcctccggccgccggaacgctcccggccggccggccgtcgatcgcgaccgcctgttccggcgggagaaccgggcggacaggggtcagagtggaaagcaccatacaagcaccttagttgaacggcccgggtgccgaacgggtcccccggccctgcaccgcgggcgaaaaataccggtcctccggggggctcggcgggccgtgcggaggcggaatcgagaggacccccaggacaggatacgaagcggcgaatggcgcgatatcgcgggtgtccggggagcggccgacgcggaacgcggcccctcacctaggctctgtcccgagggccgccccgcccggcccgggaggactccgggattcccgggaatcccggagaaccgccggagccccgggcgcatctttccccggggagccggaggacggcgggggccggggccgccgggaaccggaagggcgggccctgtgggctccgtcacttccgtacgctgggacggacggccggacagccgggcgggaagccggaccggacgccggaccggaccggcgggggcgcccggccggggcaccgagggcgacgagggcacacagcagggggcagcagatgagcaatatcccggagaccggccggactccgagggtccggatacggctcgtggtcgtccagatactggtggtctccctgctgctgaccctcggcggccggctctggcacctccagatccgggagggcgagcggtacgcggagaaggcgtcggggaaccgggtccagcgggtcgtccagcccgccgtgcgcggcgccatcctcgacgcgcgcggggccccgatcgcggacaacgagacccggctggtcgtctccgcctcccggaccgagctgctgaaggcgccggaccggggcagggccgtgctgacccggctcgccggggtcctcggcctgcccgcgcgggagctgatggacaaggtccggctgtgcgacagcaagaccccccggccctgctggaacggctcgccgtaccagcccatcccgctcaccgacgaggccaccgcccggcaggcgctccagatccgcgagcgctccgagcagttccccggcatcaccgccgagcccaccgccgtgcgccgctaccccggccccggcggcgccaacaccgcgcaggtactcggctacctctcccccgtcaccgacgaggaactgcgggccgcgcagacgtccgactccccctacctccgctccgaccaggtggggcgcagcggtctggagcggcagtacgacgacatcctgcgcggcagggcgggcatcaccgaatacgaggtcgaccatctcggccgggtcatcggccgctccggcgacacccccgcgacccccggcgccaacctgatcaccagcatcgacgcccgggtgcagcggatcgccgagtacgagctgaacgaggccatgaaggccgcccgcaaggactgggaccgcaacacccgcaggaactacgaggccgactccggcgcggtcgtcgtgatggaggcgaggacgggccgcatcgtcgccatggcgtccaatccgtcctacgaccccaacgcctgggtcggcggcatatccgccaaggactaccggaagctcaccggtgagaactccaaccacccgctgctgaaccgggcgatccagggcgtggcggcccccggctcgatcttcaaggtcataccgacggtggccgccgtcaacgcgggctaccccttcgacgggcggtacgagtgctccagctcgtactcgatcggcggccggtccttcgggaacttcgagtccaaggcgtacggccccatcgacctcggccgcgccctggaggtctcctgcgacaccgtgtactaccgcctcgcccacgaggagtggcgcaaggacggcggcctcaccccgaagaagggcgcgaaggactggttctacaggaccgcccaccagttcggcctcgggaagacgaccggcgtcgacctgccgaacgaggtcaccggccgggtccccgaccaccggtggaagcaggagtactgggaggcgaacaaggactcctggtgcgagcacggcaagcgcgacggcaccatgctggagcagttctcctgggagaactgccgccagggcaaccaggtccgcgccggtgactccgtgaactactccatcggccagggcgacaccctcaccacccccatccagatggcgacgatctacgcggccctcgccaacggcggcaccctctacacgccgagcgtgggcaaggcggtcgtcagcgccgacggccggaagatccgggagatcgagccgaaggcgcacgggaagctgcccatggaccgggtgacccgcgacaagatggaccaggccctggccggggtcgcgaccgcccggggcacggcgggctggcggttcgtcggctggccgcaggagcagatcccgatccgggccaagacgagcaccgccgaggtcttcggcaagcagaccacctcgtggttcgccacctacaccgacgactacgcggtcgttctgacgatctcccagggcggtacgggctccggcggctccgcccccgccgtccgcaagatctacaacgcgctgtacggcgtcgcgtagagtacggcaccatcgaccgttcccgggcgctgctccccaccccgcagcggaagctgcccgcgatccggcccgacggctccatcgacgcgccgaggatcc

SEQ ID NO: 2 - DNA, 1020 bp, Streptomyces clavuligerus
atgagcgtcagagaccggctcctcgcctgcttcgtggcggtgctgtggggcctgaacttcctcgccgtgcgcatcggcctcgactactaccctccggtgttcctctccgcgatgcggttcgtggtcgtggccgtgccggtgatcctcttcgtgccccggcccaaggtgcccctgcgctggctgctggtgtacggactgggcttcggtgtgatccagttcgggctgctcttcctcgccatcgacatcggcatgccctcggggcaggcgtccgtggtggtgcaggcggcggccccgttcaccatgctgctcgggctgctgctgggggagcgcatctcccggcggcaggcgggcggcatcgtgctggccgtggccgggatgagcgccatcgcggtggaacgggcgcaggacgcggccctgctgccgctggtgctgaccctggtggcggcgttcggctgggcgatgggcaatatcgcgagccggcaggcccgcccggaccatccgctgcgcttcgcgctctggatgtgtgtgctgccgccgatcccgctgctgggcctgtcggccgccctggagggcccgacggcgggctggcgggccctcggcgactcgctcacctccggcgacctcaccggtccggtggcgctgctgtacacggccctcgcgggctccgtggtcggctccgggatctggaacaccctgatgaaacggtacgaggcgggcaccgtggcgccgttctcgatgctggttccggtggtcgccgtcgcggtcgccacgatctggctggacgagcggctgaccctctggtcggcggtgtccggcgcggccgtggtgctgggcgtgctggtggggacgacccggtcgcgcgcggcccgggaggcgggcccgcccccggccgccggaaggccgccgaaggccccggcggaacgcggccggatgctgcggcggcgcccaccggggcccggggacacgggtcccgatcagtcggacggaccggcctcggcggtccagaacacccagcggtcg

SEQ ID NO: 3 - DNA, 1017 bp, Streptomyces clavuligerus
atgaacgacaccgccggcgaactggagttcgtccccctcgcggcgaacgacgacgaaacggtcggccagtggctcgacctgatggccctcgccgccgagaccggcccccgggccgcgcccccctgcaatgtcgacatggtcggctcgctgcgcttcgccccgcccgcgaccgccctcgacgactgggtcgtccgcagcgggggccgggtggtgggcgcgctgcggctggccctgcccgacggcgccccgaccgcccgcgtcgaccagctcctggtccaccccgggcggcgccgccgcggcatcggccgcgcgctctgggcccacgcccgggaactggcccgcaagcacgaccggaccaccctcaccgcgaccgtcgtcgagtccctgccctccggcccggcccaggacccgggcccggccgccttcgccgccgcgatgggcgcgcaccgctcggacatcccggcgggcacccaccagtggctcgacctcgaccggcacgacccgctggcggacggcgtccccgcggtccccgccgggtactccctcgtcacctggggcaccatcacccccgacgagtacgcggtgccggtctccgagctggaactctcgctcggcgccgggcccgtcgacagggccgcgcaggaggtcaggacgagctacgcccggcagttcgagaccatgcgggtcggccgcggccggcgggcgtaccacaccggcgccgtccacgacgccaccggcgctctcgccgggtacaccagcgtctccaagaccaccgggaaccccgcgtacgcgctccagggcatgaccgtggtgcaccgtgagcaccgcggccacgccctgggaaccctgctgaagctggccaatctggagtacgtgctgcggcacgagccggaggtgcggctcgtggagacggcgaacgccgaggacaaccacccgatgatcgccgtcaacgccgcgctgggcttcgagccctacgaccgctgggtgttctggaccgccgaggccggtccgtccgac

SEQ ID NO: 4 - DNA, 1686 bp, Streptomyces clavuligerus
atgaccaccgccgcgcgacgaccggccccgacgaccgccggagccggctgggacgccggggtgggggcgctggtgaacccctcgcggcggcgcggcgggaccctgcggctcgtctcctcggccgacgtcgactccctcgacccggcccgcacctactacgtctgggtgtggctgctccagcggctgctgaaccgtacgctcatggcctatccgaccgaccccggccccgccggactggtcccggccccggacctggccgagggccccggcgaggtcagcgacggcggccggacctggacctaccggctgcgccgggggctgcgctacgacgacggcaccccgatcacgtcggacgatgtgcgccacgccgtgcagcgggtgttcgcgcaggacgtgctgcccggcggccccacctatctcatcccgctgctggacgaccccgagcggccctaccccggtccgtaccggacggacgagccgctgcggtcggtcctcacccccgacgagcacacgatcgtcttccggctgacccggccgttctccgacttcgaccatctgatggcccagccctgcgcggcgcccgtgccccggcgctcggacaccggcgccgactacggccgcgacccgcgctcgtcgggcccctaccgggtcgcccgccatgagccggacaccctgctccacctggagcgcaatccgcactgggaccgggcgacggacccgatcaggcccgcgctgccggaccgggtggagctgaccatcggtctggacgtcgatgtcctggacgcccggctgatcgccggggagttcgacatcaacctggagggccgggggctccagcacgcggcccagcgccgggccaccgcggacgaggtgctgcgctcgcacacggacaacccccggacgagcttcctccacttcgttgcgatgcagccgcacatccccccgttcgacaatgtgcacgtacgccgcgcggtgcagtacgcggcggacaagatcctgctccaggacgcccgcggcggcccggtcaacggcggcgacctgaccacggcgctcttcccgcccaccctccccgcccaccaggacctggatctgtaccccacggggccggacctccgcggcgacctggacgcggcgcgggccgagctggcggcggcgggcctcccggacggcttccgggcggtgatcggcacccagcggggcaagttccggctggtcgccgacgcggtcgtggagtcgctcgcccgggtcggcatcgagctgaccgtcaaggaactcgacgtcgccacctacttcagcctcggcgccgggcaccccgagaccgtccgggagcacgggctcggactcctcgtcaccgactggggcgccgacttccccaccgagtacggcttcctcgccccgctcgtggacggccgccagatcaagcgcaacggcggcaactggaacctgcccgagctggacgaccccgaggtcaacgcgctgatcgacgagaccctgcacaccaccgaccccgccgcgcgcgcggagctgtggcgggccgtggagcgccgggtgatggagcacgccgtgctgctgcccctcgtccacgacaagacgctgcacttccgcaacccgtgggtcaccaacgtctatgtccacccggccttcggcctctacgacatccaggcgatgggcctcgccgaagaagac

SEQ ID NO: 5 - DNA, 1203 bp, Streptomyces clavuligerus
gtgagcggagacgccaccctgccggtgacggacgcgctgccgcacacggcggcggacgcggtgacggacgtggtggcggtgcccgacgccgccgccctccccaccagcggctggccggatctcgtcggcccggaggacttcttccagacgccccgctggctggcggtgcaggagcgcaactccggcaccacgatggacttcctggtccgccgccgcgcgggccgtcccgtcgcggccctggtgaccgcgtggggcgacgactccgtgccctggctgctggcccgcccggacgcgatgctcacccgggcgctggagggcgagggggagcaccccgaggccgaggcccggctcgccgacacggccgggggagaccccgcctcgctgctgccctcgctggtgtgcggcggccggcatctgggccgcacccgcccgctcgccgcgcccgacgcgctgccgtccgacgtggaggcgctggtcgccgaggccgagcggctcgcgaaggagcggggcgcggccaccgtctgcttcccgcacgtcgacgaccgcgacggcgccctgatcgggctgctgcgctcgcgcggctatgtctcgcacccctcggcgcactacgcctggctcgacatcccgcccggcggctgggacgagttcctggccgacatgaccaagcaccgccgccgccgggtccggctggagcgccgggcgctggacgcggcgggcatcgaggtgcggatcgagccgctgacccaggagatcgtgccccggctcggcgaactcgactgcaatctgctgcgcaagtacggcaacccggcgagccccgagcactccgcggggctgctgagctggatcgcccaggtcatgggcgacgacgtgatggtctccgtggcccgcaaggacggggtgatcatcggcttcggcatggtgctgcgctcccgggcccggggccgcgaggagtggttcggccaccgcgccggattcgactacgaggcccagggcaggctgccgctctactacgacgtcctgtactaccgggtgctggaggccgccgcccgcgagggcgtgaccgtgctgcacgccgggatcggcagcgtcgaggcgaagctggcccgcggctgtctggccagccaggagcactcgttcctgctgcgcctgccgcgcaccggggagaccggcaccaccgacaccgatcagaagaggaccgcacca

SEQ ID NO: 6 - DNA, 1311 bp, Streptomyces clavuligerus
atgaccacacccctggccgactatgccaagttcgcgggcaagcgcatcgcgatcgtcgagcacatgcagaaccaccccttcgtcctggggctccaccaggcacgtgagcacggccttgaggtctggctgctcaccggggaccggtcctggtacacccacgggcacgactgggacagccaccccctcgcggcggccgtcgaccgggtggtcgacgtggacaccaccgatctggcggccgtgctcgcggccgtcaccgacgagagcgggcagtgcctggtcgacgggctcacctcgttctccgactaccacacggtcatcgccgcgcaggcggcccagcggctgtcgctgcccggccccgaccccgaggccgtggagacggcgaacgagaaggaccggctgcgcctcgccctcggcgacgtcccctacaacatcccgcacgcccgggtcagcagccgcgagcagctcgacgaggccgtacggaagctcggcttccccatgatcgccaagccgcccgccgaggccatcagctacggggtgcgccgcgtcgacaccatggcggagctggtcgaggcgtacgaggagctgtccggcgtccgccacagcctgcgcggccagccgcgctccggcgatgtcctcctggagacctatgtcgagggcgtcgaggtcagcgtcgagacgatgaccgtggacgggtacacccatgtcttcggggtcacctccaaggacctgttcggcgaccccgcgtacatcgagtgcgggcacaccttcccggtgcccctctccgacgaggagcgcgaggcgctgtacgccgccgtccggggcaccctggaggcgatcggctaccggcacggcccctgccacaccgaggtgcggcgcaccgagaccggctggcggatcatcgaggccaacccgcgcaccccgtcgagctgtatgacgatgctggtcaccgatgtcaccgggcgcagcccgatcctcgacgcctggctgctgacgatcggcgaggccccgccgatcgagcccgtcgtccacaacggcggcgccgccgtgcgcatgatctacccgacccggaagggcaccctcacccgggtcgacggcatcgaggccgcggccgcggtgcccggggtccaggtgctgctgcacgtggagaagggcgacaagctgctccagcgcatggacaactcctcgtgcgtcggcttcacctactgcgccggggcggaccgggaggaggcccaggccctcgcggaccgcgcggccggatacctggacttcgtcgtcgaggaggacgagcggggcgagcggggcggggagtcg

SEQ ID NO: 7 - DNA, 1482 bp, Streptomyces clavuligerus
atgaacaggccgctgcggcacatagccatcttctgcgggctgctgacactcgcgctgctgctgagagccaactggctgcagtacgtggacgccgaggaactggccacgcacgagcgcaaccgccgggtccagatcaaccagttcgcgaccccgcgcggcgacatcatcgtgggtggcggtacctccatcaccgggtcgaaggccgtcgacggcaccgacttcaagtaccagcgcacctacaagaacggccccatgtacgcgcccgtcaccgggtacgcctcgcagtccatgggcatgtcgctgctggagaacacgttcgacagcgtattgagcgggcaggacgaccggctcgcgatccagcgcttcaccgacgtcgtcaccggcaaggaccggcgcggcggcaatgtgatcaccacgatcgaccccaaggcgcagcaggccgcgtacaaggggctcaccgccctggacgcccggggctcggccgtggcgctcgacccgaagacgggcaaggtgctcgccctggtctcgaccccgtcctacgaccccggtgtcttcgcgggcaacagcctcaaggagagcaagcggttccgggacctggacaaggacaagggcaagccgctggccaaccgcgcgctgcgggagaccttcccgcccggctccacgttcaagatcctcacggcggcggcggccctggagcacggggtgatcgacgacatcgacgcgaagaccgacgcggtgtccccgtatccgctgccgcagtcgaccaacacggtcggcagcgaggcgggcaacgcggcctgcgacaaggtctcgctgaagaccgccatgcagcattcctgcaacaatgtcttcctcgacgccgcggtcaagctgggcgtggacaagatgcgcgacaccgccgagaagttcgggttcaacgaggacgtctactcggacgccttcggcgacatgcgggccgtcaagagcctgtaccccaaggaactggacaagccgggcaccgcgctgaccggtatgggacagggcagcctcaccagcacccccttgcagatggccatggtcaccgccgccatcgccaacaacggcaagctgatgcagccgtacatcgtcgacgaggtgcagtcccccgatctgagcaccctggagaagaacgagccccgggagatgagccaggccgtctccgaggagaccgcgggcaaggtccgggagatgatggagttcaccgccagggagggcagcgcccgcaaggcgctgatcgacggcgtcacggtgggcggcaagaccggtaccgcgcagcgcggtgtcgatgtgcgcgagcaggtcccgtacggctggttcgtctcctacggggagaaggacggccagtccgtggcggtggccgtcttcatcgacccgaccgcgatggacatctcccgtgaggacatctccggcggcggtctgggcgcgcccatcgcccgcagtgtgatgaaggccgtcctggagaag

SEQ ID NO: 8 - protein, 340 aa, Streptomyces clavuligerus
MSVRDRLLACFVAVLWGLNFLAVRIGLDYYPPVFLSAMRFVVVAVPVILFVPRPKVPLRWLLVYGLGFGVIQFGLLFLAIDIGMPSGQASVVVQAAAPFTMLLGLLLGERISRRQAGGIVLAVAGMSAIAVERAQDAALLPLVLTLVAAFGWAMGNIASRQARPDHPLRFALWMCVLPPIPLLGLSAALEGPTAGWRALGDSLTSGDLTGPVALLYTALAGSVVGSGIWNTLMKRYEAGTVAPFSMLVPVVAVAVATIWLDERLTLWSAVSGAAVVLGVLVGTTRSRAAREAGPPPAAGRPPKAPAERGRMLRRRPPGPGDTGPDQSDGPASAVQNTQRS

SEQ ID NO: 9 - protein, 339 aa, Streptomyces clavuligerus
MNDTAGELEFVPLAANDDETVGQWLDLMALAAETGPRAAPPCNVDMVGSLRFAPPATALDDWVVRSGGRVVGALRLALPDGAPTARVDQLLVHPGRRRRGIGRALWAHARELARKHDRTTLTATVVESLPSGPAQDPGPAAFAAAMGAHRSDIPAGTHQWLDLDRHDPLADGVPAVPAGYSLVTWGTITPDEYAVPVSELELSLGAGPVDRAAQEVRTSYARQFETMRVGRGRRAYHTGAVHDATGALAGYTSVSKTTGNPAYALQGMTVVHREHRGHALGTLLKLANLEYVLRHEPEVRLVETANAEDNHPMIAVNAALGFEPYDRWVFWTAEAGPSD

SEQ ID NO: 10 - protein, 562 aa, Streptomyces clavuligerus
MTTAARRPAPTTAGAGWDAGVGALVNPSRRRGGTLRLVSSADVDSLDPARTYYVWVWLLQRLLNRTLMAYPTDPGPAGLVPAPDLAEGPGEVSDGGRTWTYRLRRGLRYDDGTPITSDDVRHAVQRVFAQDVLPGGPTYLIPLLDDPERPYPGPYRTDEPLRSVLTPDEHTIVFRLTRPFSDFDHLMAQPCAAPVPRRSDTGADYGRDPRSSGPYRVARHEPDTLLHLERNPHWDRATDPIRPALPDRVELTIGLDVDVLDARLIAGEFDINLEGRGLQHAAQRRATADEVLRSHTDNPRTSFLHFVAMQPHIPPFDNVHVRRAVQYAADKILLQDARGGPVNGGDLTTALFPPTLPAHQDLDLYPTGPDLRGDLDAARAELAAAGLPDGFRAVIGTQRGKFRLVADAVVESLARVGIELTVKELDVATYFSLGAGHPETVREHGLGLLVTDWGADFPTEYGFLAPLVDGRQIKRNGGNWNLPELDDPEVNALIDETLHTTDPAARAELWRAVERRVMEHAVLLPLVHDKTLHFRNPWVTNVYVHPAFGLYDIQAMGLAEED

SEQ ID NO: 11 - protein, 401 aa, Streptomyces clavuligerus
VSGDATLPVTDALPHTAADAVTDVVAVPDAAALPTSGWPDLVGPEDFFQTPRWLAVQERNSGTTMDFLVRRRAGRPVAALVTAWGDDSVPWLLARPDAMLTRALEGEGEHPEAEARLADTAGGDPASLLPSLVCGGRHLGRTRPLAAPDALPSDVEALVAEAERLAKERGAATVCFPHVDDRDGALIGLLRSRGYVSHPSAHYAWLDIPPGGWDEFLADMTKHRRRRVRLERRALDAAGIEVRIEPLTQEIVPRLGELDCNLLRKYGNPASPEHSAGLLSWIAQVMGDDVMVSVARKDGVIIGFGMVLRSRARGREEWFGHRAGFDYEAQGRLPLYYDVLYYRVLEAAAREGVTVLHAGIGSVEAKLARGCLASQEHSFLLRLPRTGETGTTDTDQKRTAP

SEQ ID NO: 12 - protein, 437 aa, Streptomyces clavuligerus
MTTPLADYAKFAGKRIAIVEHMQNHPFVLGLHQAREHGLEVWLLTGDRSWYTHGHDWDSHPLAAAVDRVVDVDTTDLAAVLAAVTDESGQCLVDGLTSFSDYHTVIAAQAAQRLSLPGPDPEAVETANEKDRLRLALGDVPYNIPHARVSSREQLDEAVRKLGFPMIAKPPAEAISYGVRRVDTMAELVEAYEELSGVRHSLRGQPRSGDVLLETYVEGVEVSVETMTVDGYTHVFGVTSKDLFGDPAYIECGHTFPVPLSDEEREALYAAVRGTLEAIGYRHGPCHTEVRRTETGWRIIEANPRTPSSCMTMLVTDVTGRSPILDAWLLTIGEAPPIEPVVHNGGAAVRMIYPTRKGTLTRVDGIEAAAAVPGVQVLLHVEKGDKLLQRMDNSSCVGFTYCAGADREEAQALADRAAGYLDFVVEEDERGERGGES

SEQ ID NO: 13 - protein, 494 aa, Streptomyces clavuligerus
MNRPLRHIAIFCGLLTLALLLRANWLQYVDAEELATHERNRRVQINQFATPRGDIIVGGGTSITGSKAVDGTDFKYQRTYKNGPMYAPVTGYASQSMGMSLLENTFDSVLSGQDDRLAIQRFTDVVTGKDRRGGNVITTIDPKAQQAAYKGLTALDARGSAVALDPKTGKVLALVSTPSYDPGVFAGNSLKESKRFRDLDKDKGKPLANRALRETFPPGSTFKILTAAAALEHGVIDDIDAKTDAVSPYPLPQSTNTVGSEAGNAACDKVSLKTAMQHSCNNVFLDAAVKLGVDKMRDTAEKFGFNEDVYSDAFGDMRAVKSLYPKELDKPGTALTGMGQGSLTSTPLQMAMVTAAIANNGKLMQPYIVDEVQSPDLSTLEKNEPREMSQAVSEETAGKVREMMEFTAREGSARKALIDGVTVGGKTGTAQRGVDVREQVPYGWFVSYGEKDGQSVAVAVFIDPTAMDISREDISGGGLGAPIARSVMKAVLEK

SEQ ID NO: 14 - protein, 573 aa, Streptomyces clavuligerus
MSRVSTAPSGKPTAAHALLSRLRDHGVGKVFGVVGREAASILFDEVDPIDFVLTRHDFTAGVAADVLARITGRPQACWATLGPGMTNLSTGIATSVLESSPVIALAAQSESHDIFPNDTHQCLDSVAIVAPMSLYAVELQRPHEITDLVDSAVNAAMTEPVGPSFISLPVDLLGSSEGIDTTVPNPPANTPAKPVGVVADGWQKAADQAAALLAEAKHPVLVVGAAAIRSGAVPAIRALAQRLNIPVITTYIAKGVLPVGHELNYGAVTGYMDGILNFPALQTMFAPVDLVLTVGYDYAEDLRPSMWQKGIEKKTVRISPTVNPIPRVYRPDVDVVTDVLAFVEHFETATASFGAKQRHDIEPLRARIAEFLADPETYEDGMRVHQVIDSMNTVMEEAAEPGEGTIVSDIGFFRHYGVLFARADQPFGFLTSAGCSSFGYGIPAAIGAQMARPDQPTFLIAGDGGFHSNSSDLETIARLNLPIVTVVVNNDTNGLIELYQNIGHHRSHDPAVKFGGVDFVALAEANGVDATRATNREELLAALRKGAELGRPFLIEVPVNYDFQPGGFGALSI

SEQ ID NO: 15 - protein, 513 aa, Streptomyces clavuligerus
MGAPVLPAAFGFLASARTGGGRAPGPVFATRGSHTDIDTPQGERSLAATLVHAPSVAPDRAVARSLTGAPTTAVLAGEIYNRDELLSVLPAGPAPEGDAELVLRLLERYDLHAFRLVNGRFATVVRTGDRVLLATDHAGSVPLYTCVAPGEVRASTEAKALAAHRDPKGFPLADARRVAGLTGVYQVPAGAVMDIDLGSGTAVTHRTWTPGLSRRILPEGEAVAAVRAALEKAVAQRVTPGDTPLVVLSGGIDSSGVAACAHRAAGELDTVSMGTDTSNEFREARAVVDHLRTRHREITIPTTELLAQLPYAVWASESVDPDIIEYLLPLTALYRALDGPERRILTGYGADIPLGGMHREDRLPALDTVLAHDMATFDGLNEMSPVLSTLAGHWTTHPYWDREVLDLLVSLEAGLKRRHGRDKWVLRAAMADALPAETVNRPKLGVHEGSGTTSSFSRLLLDHGVAEDRVHEAKRQVVRELFDLTVGGGRHPSEVDTDDVVRSVADRTARGAA

SEQ ID NO: 16 - protein, 313 aa, Streptomyces clavuligerus
VERIDSHVSPRYAQIPTFMRLPHDPQPRGYDVVVIGAPYDGGTSYRPGARFGPQAIRSESGLIHGVGIDRGPGTFDLINCVDAGDINLTPFDMNIAIDTAQSHLSGLLKANAAFLMIGGDHSLTVAALRAVAEQHGPLAVVHLDAHSDTNPAFYGGRYHHGTPFRHGIDEKLIDPAAMVQIGIRGHNPKPDSLDYARGHGVRVVTADEFGELGVGGTADLIREKVGQRPVYVSVDIDVVDPAFAPGTGTPAPGGLLSREVLALLRCVGDLKPVGFDVMEVSPLYDHGGITSILATEIGAELLYQYARAHRTQL

SEQ ID NO: 17 - protein, 325 aa, Streptomyces clavuligerus
MASPIVDCTPYRDELLALASELPEVPRADLHGFLDEAKTLAARLPEGLAAALDTFNAVGSEDGYLLLRGLPVDDSELPETPTSTPAPLDRKRLVMEAMLALAGRRLGLHTGYQELRSGTVYHDVYPSPGAHYLSSETSETLLEFHTEMAYHILQPNYVMLACSRADHENRAETLVGSVRKALPLLDEKTRARLFDRKVPCCVDVAFRGGVDDPGAIANVKPLYGDANDPFLGYDRELLAPEDPADKEAVAHLSQALDDVTVGVKLVPGDVLIIDNFRTTHARTPFSPRWDGKDRWLHRVYIRTDRNGQLSGGERAGDTISFSPRR

SEQ ID NO: 18 - protein, 393 aa, Streptomyces clavuligerus
MSDSTPKTPRGFVVHTAPVGLADDGRHDFTVLASTAPATVSAVFTRSRFAGPSVVLCREAVADGQARGVVVLARNANVATGLEGEENAREVREAVARALGLPEGEMLIASTGVIGRQYPMESIREHLKTLEWPAGEGGFDRAARAIMTTDTRPKEVRVSVGGATLVGIAKGVGMLEPDMATLLTFFATDARLDPAEQDRLFRRVMDRTFNAVSIDTDTSTSDTAVLFANGLAGEVDAGEFEEALHTAALALVKDIASDGEGAAKLIEVQVTGARDDAQAKRVGKTVVNSPLVKTAVHGCDPNWGRVAMAIGKCSDDTDIDQERVTIRFGEVEVYPPKARGDQADDALRAAVAEHLRGDEVVIGIDLAIADGAFTVYGCDLTEGYVRLNSEYTT

SEQ ID NO: 19 - protein, 555 aa, Streptomyces clavuligerus
METTRSTTADEGFDAGVRGVVAPTDAPGGTLRLVRTDDFDSLDPGNTYYAYTWNFLRLIGRTLVTFDTAPGKAGQRLVPDLAESLGESSEDGRVWTYRLREGLRYEDGTPVVSADIKHAIARSNYGTDVLGAGPTYFRHLLGTEYGGPWREPDADGPVTLETPDERTLVFRLREPFAGMDLLATMPSTTPVPRDRDTGAEYRLRPVATGPYRIVSYTRGELAVLEPNPHWDPETDPVRVQRASRIEVHLGKDPHEVDRMLLAGEAHVDLAGFGVQPAAQERILAEPELRAHADNPLTGFTWIYCLSSRIAPFDNVHCRRAVQFATDKAAMQEAYGGAVGGDIATTLLPPTLDGYKHFDRYPVGPEGTGDLEAARAELKLAGMPDGFRTRIAARKDRLKEYRAAEALAAGLARVGIEAEVLDFPSGDYFDRYGGCPEYLREHGIGIIMFGWGADFPDGYGFLQQITDGRAIKERGNQNMGELDDPEINALLDEGAQCADPARRAEIWHRIDQLTMDHAVIVPYLYPRSLLYRHPDTRNAFVTGSFGMYDYVALGAK

SEQ ID NO: 20 - protein, 432 aa, Streptomyces clavuligerus
MEVARRTGVRHGTVERRLDRLDRIVGLPLTLRSRHTARLTTAGSRILVAGRRFFHQVDLAARTHIFGHGSEAVDAPEVLSLVSTEPLLDEVVEDAAASLDLLLSVRHEAPHQVAAQLAGYQVDAAYTWSLQSPRHSLERSVRTCEVLDDPLWVILPRDHPLAARREVSLADLRDETWVSETGPGSEILVTRVFQLAGLTAPTRLHITGASVARGILRRGDAIGLGSPTHPAVQDPSLVRRSLAERPRRTTSLLVDPTIVPRALAGRLAALIAEVQLRRFAEHHRDLLDEPWWAQWYAERTGADARRFGAGPDQGSVPGQAEGRKLDVDDLHLLQAVARHGSINRAAAVLSISQSALTRRIHRLEQSLGARLLLRSPRGTSLTGPTRQFLRQLALYEAEFREAALACRSVERPLAQGHWPIRRGVAAGARMSG

SEQ ID NO: 21 - protein, 247 aa, Streptomyces clavuligerus
MPSALQGKVALITGASSGIGEATARALAAEGAAVAIAARRVEKLRALGDELTAAGAKVHVLELDVADRQGVDAAVASTVEALGGLDILVNNAGIMLLGPVEDADTTDWTRMIDTNLLGLMYMTRAALPHLLRSKGTVVQMSSIAGRVNVRNAAVYQATKFGVNAFSETLRQEVTERGVRVVVIEPGTTDTELRGHITHTATKEMYEQRISQIRKLQAQDIAEAVRYAVTAPHHATVHEIFIRPTDQV

SEQ ID NO: 22 - protein, 408 aa, Streptomyces clavuligerus
MMNEAAPQSDQVAPAYPMHRVCPVDPPPQLAGLRSQKAASRVTLWDGSQVWLVTSHAGARAVLGDRRFTAVTSAPGFPMLTRTSQLVRANPESASFIRMDDPQHSRLRSMLTRDFLARRAEALRPAVRELLDEILGGLVKGERPVDLVAGLTIPVPSRVITLLFGAGDDRREFIEDRSAVLIDRGYTPEQVAKARDELDGYLRELVEERIENPGTDLISRLVIDQVRPGHLRVEEMVPMCRLLLVAGHGTTTSQASLSLLSLLTDPELAGRLTEDPALLPKAVEELLRFHSIVQNGLARAAVEDVQLDDVLIRAGEGVVLSLSAGNRDETVLPDPDRVDVDRDARRHLAFGHGMHQCLGQWLARVELEEILAAVLRWMPGARLAVPFEELDFRHEVSSYGLGALPVTW